(19) United States
(12) Patent Application Publication
(10) Pub. No.: US 2004/0076668 A1
(43) Pub. Date: Apr. 22, 2004

(54) Controlled-release pharmaceutical formulations

(75) Inventors: Alfred Berchielli, Niantic, CT (US); Patrick D. Daugherity, Niantic, CT (US); Sheri L. Shamblin, North Stonington, CT (US); Avinash G. Thombre, East Lyme, CT (US); Kenneth C. Waterman, East Lyme, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/608,817

(22) Filed: Jun. 25, 2003

Related U.S. Application Data
(60) Provisional application No. 60/393,801, filed on Jul. 3, 2002.

(51) Int. Cl. A61K 31/4433; A61K 9/22

(52) U.S. Cl. 424/468; 514/355; 514/338

(57) ABSTRACT

The invention provides controlled release pharmaceutical formulations having a coated core with the core comprising a drug-containing composition and a water-swellable composition, each occupying essentially separate regions within the core. The drug-containing composition comprises a PDE4D inhibitor, or a pharmaceutically acceptable salt thereof, preferably (R)-2-[4-({[2-(benzo[1,3]dioxol-5-yloxy)-pyridine-3-carbonyl]-amino}-methyl)-3-fluoro-phenoxy]-propionic acid, or a pharmaceutically acceptable salt thereof, or 2-(4-fluorophenoxy)-N-[4-(1-hydroxy-1-methyl-ethyl)-benzyl]-nicotinamide, or a pharmaceutically acceptable salt thereof, and a drug-entraining agent. The coating around the core is water-permeable, water-insoluble, and has at least one delivery port therethrough. The invention further provides methods of reducing the nausea and emetic effects of PDE4D inhibitors, and controlled-release formulations having improved PDE4D inhibitor in vivo and in vitro profiles.

This application is filed claiming priority from co-pending U.S. Provisional Application No. 60/393,801, filed Jul. 3, 2002.

### BACKGROUND OF THE INVENTION

The invention relates generally to controlled-release pharmaceutical formulations providing a controlled release of a beneficial agent to an environment of use.

Specifically, the controlled-release pharmaceutical formulations of the instant invention utilize so-called “swellable-core technology” (SCT). Such technology is disclosed in commonly-assigned PCT International Application Publication No. WO 01/47500, the disclosure of which is incorporated herein by reference in its entirety. Formulations comprising SCT generally incorporate a core comprising a drug-containing composition and a water-swellable composition. The drug-containing composition and the water-swellable composition occupy separate regions within the core. The drug-containing composition comprises a low-solubility drug and a drug-entraining agent. In accordance with the controlled-release formulations and methods of the present invention, preferred drugs comprising the drug-containing composition are PDE4D inhibitors. A coating surrounding the core is water-permeable, water-insoluble, and has at least one delivery port therethrough. The core imbibes water through the coating which causes the water-swellable composition to expand, thereby increasing pressure within the core. The pressure gradient between the core and the environment of use, i.e., preferably the gastrointestinal (GI) tract of the patient, drives the release of the drug-containing composition, which is extruded out of the core through the delivery port(s) into the environment of use. Because the water-swellable composition contains no drug, virtually all of the drug in the drug-containing composition is extruded through the delivery port(s) into the environment of use, leaving very little residual drug inside the formulation.

The preferred PDE4D inhibitors, and the pharmaceutically acceptable salts thereof, useful in the controlled-release formulations and methods of the present invention will be well known to one of ordinary skill in the art. A number of selective inhibitors of PDE4D have been discovered recently, and beneficial pharmacological effects resulting from that inhibition have been demonstrated in a number of disease models. See, e.g., Torphy, et al., Environ. Health Perspect., 102, Suppl. 10, 79-84 (1994); Duplantier, et al., J. Med. Chem., 39, 120-125 (1996); Schneider, et al., Pharmacol. Biochem. Behav., 50, 211-217 (1995); Banner and Page, Br. J. Pharmacol., 114, 93-98 (1995); Barnette, et al., J. Pharmacol. Exp. Ther., 273, 674679 (1995); Wright, et al., Can. J. Physiol. Pharmacol., 75, 1001-1008, (1997); Manabe, et al., Eur. J. Pharmacol., 332, 97-107 (1997); and Ukita, et al., J. Med. Chem., 42, 1088-1099 (1999). PDE4 inhibitors are known to be useful in the treatment of a number of inflammatory, respiratory, and allergic disorders and conditions mediated by the PDE4 isozyme including, but not limited to, asthma; chronic obstructive pulmonary disease (COPD), including chronic bronchitis, emphysema, and bronchiectasis; chronic rhinitis; and chronic sinusitis. Within the airways of patients suffering from asthma and other obstructive airway diseases, PDE4 is the most important of the PDE isozymes as a target for drug discovery because of its ubiquitous distribution in airway smooth muscle and inflammatory cells. Airflow obstruction and airway inflammation are features of asthma as well as COPD. Thus, PDE's such as PDE4 that are involved in smooth muscle relaxation, and are also found in eosinophils as well as neutrophils, are believed to constitute an essential element in the etiology of both diseases.

Although many PDE4 inhibitors introduced to the art have been designed to have reduced gastrointestinal and central nervous system side-effects, nausea and emesis, i.e., vomiting, particularly PDE4 inhibitors selective towards the subtype D, or PDE4D inhibitors, some PDE4D inhibitors continue to present in many patients being treated with such inhibitors. The mechanism(s) by which PDE4D inhibitors induce nausea and/or emesis is/are presently unknown, however, it is currently believed that such effects are at least partially mediated by emesis centers in the brain, and/or by local gastrointestinal disturbance.

The present invention provides improved release profiles for the preferred PDE4D inhibitors to an environment of use and a means of achieving such profiles through the use of SCT controlled-release formulations. Such improved release profiles afford PDE4 formulations allowing once or twice daily dosing regimens, with concomitant significant reduction in both the nausea and emesis induced by the administration of such inhibitors.

### SUMMARY OF THE INVENTION

The invention provides improved release profiles for the preferred PDE4D inhibitors to an environment of use. We have shown that when pharmaceutical formulations of PDE4D inhibitors are administered, the threshold for nausea and emesis are not simply related to the plasma concentrations of the PDE4D inhibitors, typically measured by the peak plasma concentration (or Cₘₐₓ), but also to the time at which these peak concentrations are reached (or Tₘₐₓ). Furthermore, the rate of rise of plasma concentrations, approximated by a Cₘₐₓ/Tₘₐₓ ratio, is a critical factor that determines the tolerability of PDE4D inhibitors and by appropriately manipulating the Cₘₐₓ/Tₘₐₓ ratio, the thresholds for typical side effects such as nausea and emesis can be increased, thereby allowing higher and more therapeutic doses to be administered.

The invention further provides controlled release pharmaceutical formulations having a coated core with the core comprising a drug-containing composition and a water-swellable composition, each occupying essentially separate regions within the core. The drug-containing composition comprises a PDE4D inhibitor, or a pharmaceutically acceptable salt thereof, preferably (R)-2-[4-({[2-(benzo[1,3]dioxol-5-yloxy)-pyridine-3-carbonyl]-amino}-methyl)-3-fluoro-phenoxy]-propionic acid, or a pharmaceutically acceptable salt thereof, or 2-(4-fluorophenoxy)-N-[4-(1-hydroxy-1-methyl-ethyl)-benzyl]-nicotinamide, or a pharmaceutically acceptable salt thereof, and a drug-entraining agent. The coating around the core is water-permeable, water-insoluble, and has at least one delivery port therethrough.

The invention further provides methods of reducing the nausea and emetic effects of PDE4D inhibitors, and controlled-release formulations having improved PDE4D inhibitor in vivo and in vitro profiles.

### DETAILED DESCRIPTION OF THE INVENTION

The present invention provides controlled-release pharmaceutical formulations comprising phosphodiesterase type 4D (PDE4D) inhibitors, or their pharmaceutically acceptable salts, thereof, wherein said formulations exhibit at least one of the following characteristics:

(i) a Tₘₐₓ of greater than about 1.5 hours;

(ii) less than about 80% of the PDE4D inhibitor, or said pharmaceutically acceptable salt thereof, is released in vivo at about 1.5 hours;

(iii) less than about 80% of the PDE4D inhibitor, or said pharmaceutically acceptable salt thereof, is released in vitro at about 1.5 hours;

(iv) an in vivo delivery time lag prior to initiation of the PDE4D inhibitor, or said pharmaceutically acceptable salt thereof, of between 0.5 hours to about 4 hours;

(v) an in vitro delivery time lag prior to initiation of the PDE4D inhibitor, or said pharmaceutically acceptable salt thereof, of between 0.5 hours to about 4 hours.

In a preferred embodiment, the present invention provides controlled-release pharmaceutical formulations comprising a core, and a coating around said core, wherein:

(a) the core comprises a drug-containing composition and a water-swellable composition, each occupying essentially separate regions within the core;

(b) the drug-containing composition comprises a PDE4 inhibitor, or a pharmaceutically acceptable salt thereof, and a drug-entraining agent;

(c) the water-swellable composition comprises a swelling agent; and

(d) the coating is water-permeable, water-insoluble, and has at least one delivery port therethrough.

A detailed description of the component elements and construction of the SCT controlled-release formulations useful in the practice of the instant invention has been previously disclosed in the aforementioned WO 01/47500. A summary of such disclosure, incorporating the preferred embodiments of the present invention, appears hereinbelow.

### The Drug-Containing Composition

The drug-containing composition of the core of the controlled-release formulations of the present invention comprises a PDE4D inhibitor, or a pharmaceutically acceptable salt thereof, and a drug-entraining agent. The drug-containing composition occupies a separate, essentially distinct region from the water-swellable composition, and comprises from about 50% to about 90 wt % of the core, preferably from about 60% to about 85 wt % of the core, and, more preferably, greater than about 70 wt % of the core. The mass ratio of the drug-containing composition to the water-swellable composition preferably has a value of at least 1.0. Preferably, the drug-containing composition is in contact with the coating that surrounds the formulation. The PDE4D inhibitor, or the pharmaceutically acceptable salt thereof, may be in any form, either crystalline or amorphous.

In one embodiment, the PDE4D inhibitor, or the pharmaceutically acceptable salt thereof, is present in the drug-containing composition in the form of a solid, amorphous dispersion. In such solid, amorphous dispersion, the PDE4D inhibitor, or the pharmaceutically acceptable salt thereof, is dispersed in a polymer such that a major portion of the inhibitor is in a substantially amorphous, or non-crystalline state. The dispersion may comprise from about 5% to about 90 wt % of the PDE4D inhibitor, preferably from about 10% to about 70 wt %. More preferably, the amorphous dispersion comprises a solid dispersion of the PDE4D inhibitor, or the pharmaceutically acceptable salt thereof, in a concentration-enhancing polymer. Suitable polymers may comprise, for example, ionizable and non-ionizable cellulosic polymers, such as cellulose esters, cellulose ethers, and cellulose esters/ethers; and vinyl polymers and copolymers having substituents selected from the group consisting of hydroxyl, alkylacyloxy, and cylicamido, such as polyvinyl pyrrolidone (PVP), polyvinyl alcohol (PVA), and copolymers of polyvinyl pyrolidone and polyvinyl acetate. Additionally preferred polymers may comprise, for example, hydroxypropylmethyl cellulose acetate succinate (HPMCAS), hydroxypropylmethyl cellulose (HPMC), hydroxypropylmethyl cellulose phthalate (HPMCP), cellulose acetate phthalate (CAP), and cellulose acetate trimellitate (CAT).

The drug-containing composition further comprises a drug-entraining agent. The drug-entraining agent suspends, or entrains, the PDE4D inhibitor, or the pharmaceutically acceptable salt thereof, so as to aid in the delivery thereof through the delivery port(s) to the environment of use. It is presently believed that upon the imbibition of water into the formulation, the drug-entraining agent imparts sufficient viscosity to the drug-containing composition to allow it to suspend, or entrain, the PDE4D inhibitor, or the pharmaceutically acceptable salt thereof, while at the same time remain sufficiently fluid to allow the entraining agent to pass through the delivery port(s) together with the PDE4D inhibitor, or the pharmaceutically acceptable salt thereof. The drug-entraining agent generally comprises a material that has high water solubility and in operation forms aqueous solutions with viscosities of at least 50 centipoise (cp), and preferably aqueous solutions with viscosities of 200 cp or greater.

The amount of the drug-entraining agent present in the drug-containing composition may range from about 20 wt % to about 98 wt % of the drug-containing composition. Preferably, the drug-entraining agent comprises at least about 15 wt % of the drug-containing composition. The drug-entraining agent may comprise a single material, or a mixture of materials. Examples of such materials include polyols, and oligomers of polyethers, such as ethylene glycol oligomers, or propylene glycol oligomers. In addition, mixtures of polyfunctional organic acids and cationic materials, such as amino acids, or multivalent salts, such as calcium salts may be used. Of particular utility are polymers such as polyethylene oxide (PEO), PVA, PVP, cellulosics such as hydroxyethyl cellulose (HEC), hydroxypropylcellulose (HPC), HPMC, methyl cellulose (MC), carboxy methyl cellulose (CMC), carboxyethylcellulose (CEC), gelatin, xanthan gum, or any other water-soluble polymer that forms an aqueous solution with a viscosity similar to that of the polymers listed above. A preferred drug-entraining agent comprises non-crosslinked PEO, or a mixture of PEO with any of the other materials denoted hereinabove. When the PDE4D inhibitor, or the pharmaceutically acceptable salt thereof, comprise about 80 wt % or more of the drug-containing composition, then the drug-entraining agent should have a sufficiently low molecular weight that it becomes sufficiently fluid so that both the PDE4D inhibitor, or the pharmaceutically acceptable salt thereof, and the drug-entraining agent can be rapidly extruded from the formulation, instead of swelling and rupturing the water-permeable coating that surrounds the formulation. When the PDE4D inhibitor, or the pharmaceutically acceptable salt thereof, and the drug-entraining agent make up less than about 80 wt % of the drug-containing composition, a smaller portion of a more viscous entraining agent is typically preferred.

In a preferred embodiment, the drug-containing composition further comprises a swelling agent. The swelling agent generally comprises a water-swellable polymer, preferably an ionic or non-ionic polymer, which substantially expands in the presence of water. Ionic polymers are generally polymers having a significant number of functional groups that are substantially ionized in an aqueous solution over at least a portion of the physiologically relevant pH range 1 to 8. Such ionizable functional groups include carboxylic acids and their salts, sulfonic acids and their salts, amines and their salts, and pyridine salts. Preferred ionic polymeric swelling agents include polacrilin potassium, polyacrylin resins, sodium alginate, sodium starch glycolate, and sodium croscarmellose. Preferred non-ionic polymer swelling agents include alginic acid, CMC, HPC, microcrystalline cellulose, crospovidone, MC, PEO, povidone, and starch. The swelling agent is typically present in an amount ranging from about 2 to about 20 wt % of the drug-containing composition.

In another preferred embodiment of the present invention, the drug-containing composition further comprises a fluidizing agent. The fluidizing agent allows the drug-containing composition to rapidly become fluid upon imbibing water when the dosage form is introduced into a use environment, thereby allowing the composition to be extruded from the dosage form without a build-up of excessive pressure inside the core. In addition, the inclusion of a fluidizing agent reduces the pressure within the core, thereby reducing the risk of failure of the coating surrounding the core. This is particularly important when a relatively rapid rate of drug release is desired, necessitating the use of a highly water-permeable coating that conventionally is relatively thin and weak. The fluidizing agent can comprise any water-soluble compound that rapidly increases the fluidity of the drug-containing composition when water is imbibed into the core. Such compounds generally have aqueous solubilities of at least 30 mg/mL and generally have a relatively low molecular weight (less than 10,000 daltons) such that upon imbibition of a given quantity of water, the drug-containing composition rapidly becomes more fluid relative to a similar drug-containing composition that does not include the fluidizing agent. The phrase “more fluid” means that the pressure required to extrude the drug through the delivery port(s) is lower than a similar composition without the fluidizing agent. This increased fluidity can be temporary, meaning that the increased fluidity occurs for only a short time after introduction of the dosage form to a use environment (e.g., 2 hours), or the increased fluidity can occur over the entire time the dosage form is in the use environment. Exemplary fluidizing agents comprise sugars, organic acids, organic bases, amino acids, polyols, salts, and low-molecular weight oligomers of water-soluble polymers. Exemplary sugars are glucose, sucrose, xylitol, fructose, lactose, mannitol, sorbitol, maltitol, and the like. Exemplary organic acids are citric acid, lactic acid, ascorbic acid, tartaric acid, malic acid, fumaric, and succinic acid. Exemplary amino acids are alanine and glycine. Exemplary polyols are propylene glycol and sorbitol. Exemplary oligomers of low-molecular weight polymers are polyethylene glycols with molecular weights of 10,000 daltons or less. Particulalry preferred fluidizing agents comprise organic acids, organic bases, and sugars.

In order for the fluidizing agent to rapidly increase the fluidity of the drug-containing composition at low water levels in the core of the formulation, the fluidizing agent is generally present in an amount such that it comprises at least about 10 wt % of the drug-containing composition. To ensure that the drug-containing composition does not become so fluid such that the drug-entraining agent cannot properly entrain or suspend the PDE4D inhibitor, or the pharmaceutically acceptable salt thereof, particularly long after introduction of the formulation into the use environment, the amount of fluidizing agent generally does not exceed about 60 wt % of the drug-containing composition. When the fluidizing agent is included, a drug-entraining agent with a higher molecular weight and correspondingly higher viscosity is generally included in the drug-containing composition, but at a lower level. Thus, for example, when the drug-containing composition comprises about 20 wt % to 30 wt % of the low-solubility drug and about 30 wt % of a fluidizing agent such as a sugar, about 20 wt % to 50 wt % of a high molecular weight polymer such as PEO with a molecular weight of about 500,000 to 800,000 daltons is preferable to a lower molecular weight PEO.

In another preferred embodiment, the drug-containing composition further comprises a solubilizer, utilized to promote the aqueous solubility of the drug, which is present in an amount ranging up to about 30 wt % of the drug-containing composition. Examples of preferred solubilizers may include surfactants; pH control agents such as buffers, organic and inorganic acids, and organic acid salts; organic and inorganic bases; mono-, di-, and tri-glycerides; glyceride derivatives; polyhydric alcohol esters; PEG and PPG esters; polyoxyethylene and polyoxypropylene ethers and their copolymers; phospholipids, such as lecithin; sorbitan esters; polyoxyethylene sorbitan esters; carbonate salts; zeolites; and cyclodextrins. Preferred surfactant solubilizers may comprise, for example, lapyrium chloride; laureth 4, i.e., α-dodecyl-ω-hydroxy-poly(oxy-1,2-ethanediyl) or polyethylene glycol monododecyl ether; laureth 9, i.e., a mixture of polyethylene glycol monododecyl ethers averaging about 9 ethylene oxide groups per molecule; monoethanolamine; nonoxynol 4, 9 and 10, i.e., polyethylene glycol mono(p-nonylphenyl) ether; nonoxynol 15, i.e., α-(p-nonylphenyl)-ω-hydroxypenta-deca(oxyethylene); nonoxynol 30, i.e., α-(p-nonylphenyl)-ω-hydroxytriaconta(oxyethylene); poloxalene, i.e., nonionic polymer of the polyethylenepolypropylene glycol type, MW=approx. 3000; polyoxyl 8, 40 and 50 stearate, i.e., poly(oxy-1,2-ethanediyl), α-hydro-ω-hydroxy-; octadecanoate; polyoxyl 10 oleyl ether, i.e., poly(oxy-1,2-ethanediyl), α-[(Z)-9-octadecenyl-ω-hydroxy-; polysorbate 20, i.e., sorbitan, monododecanoate, poly(oxy-1,2-ethanediyl); polysorbate 40, i.e., sorbitan, monohexadecanoate, poly(oxy-1,2-ethanediyl); polysorbate 60, i.e., sorbitan, monooctadecanoate, poly(oxy-1,2-ethanediyl); polysorbate 65, i.e., sorbitan, trioctadecanoate, poly(oxy-1,2-ethanediyl); polysorbate 80, i.e., sorbitan, mono-9-monodecenoate, poly(oxy-1,2-ethanediyl); polysorbate 85, i.e., sorbitan, tri-9-octadecenoate, poly(oxy-1,2-ethanediyl); sodium lauryl sulfate; sorbitan monolaurate; sorbitan monooleate; sorbitan monopalmitate; sorbitan monostearate; sorbitan sesquioleate; sorbitan trioleate; and sorbitan tristearate. The preferred cyclodextrin solubilizers will be well-known to one of ordinary skill in the art, and comprise a family of natural cyclic oligosaccharides capable of forming inclusion complexes with a variety of materials. Preferred cyclodextrins may comprise, for example, those having 6-, 7-, and 8-glucose residues in a ring, commonly referred to as α-cyclodextrins, β-cyclodextrins, and γ-cyclodextrins, respectively. Especially preferred cyclodextrins comprise α-cyclodextrin, β-cyclodextrin, and y-cyclodextrin, δ-cyclodextrin, and cationized cyclodextrins.

The preferred solubilizer for a specific PDE4D inhibitor is dependent on the physicochemical properties such as salt form, intrinsic solubility and pKa, i.e., the pH-dependent solubility of the PDE4D inhibitor, and will be obvious to one skilled in the art. A preferred class of solubilizers for basic PDE4D inhibitors comprises organic acids. A generally preferred subset of organic acids comprises citric, succinic, fumaric, adipic, malic and tartaric acids. Exemplary classes of solubilizers for acidic PDE4D inhibitors comprise alkylating agents, buffering agents, and organic bases. Preferred examples of alkylating or buffering agents include potassium citrate, sodium bicarbonate, sodium citrate, dibasic sodium phosphate, and monobasic sodium phosphate. Examples of organic bases include meglumine, monoethanolamine, diethanolamine, and triethanolamine.

In yet another preferred embodiment, the drug-containing composition further comprises a concentration-enhancing polymer that enhances the concentration of the PDE4D inhibitor, or the pharmaceutically acceptable salt thereof, in an environment of use relative to control compositions that are free from the concentration-enhancing polymer. Especially preferred polymers are those disclosed hereinabove for forming solid-amorphous dispersions of the PDE4D inhibitor with a polymer. Preferred polymers include HPMCAS, HPMC, HPMCP, CAP, CAT, and PVP.

The drug-containing composition may further comprise excipients that promote drug stability. Examples of such stabilizing agents include pH control agents such as buffers, organic acids and organic acid salts, and organic and inorganic bases and base salts. These excipients can be the same materials listed above for use as solubilizers or fluidizing agents. Another class of stabilizing agents comprises antioxidants, such as butylated hydroxytoluene (BHT), butylated hydroxyanisole (BHA), vitamin E, and ascorbyl palmitate. The amount of stabilizing agent used in the drug-containing composition should be sufficient to stabilize the PDE4D inhibitor, or the pharmaceutically acceptable salt thereof. For pH control, agents such as organic acids, the stabilizing agent, when present, may range from about 0.1 wt % to about 20 wt % of the drug-containing composition. The amount of antioxidant used in the drug-containing composition generally ranges from 0 wt % to about 1 wt % of the drug-containing composition.

Finally, the drug-containing composition may further comprise additional conventional excipients, such as those that promote performance, tableting, or processing of the formulation. Such excipients include tableting aids, surfactants, diluents, water-soluble polymers, pH modifiers, fillers, binders, pigments/dyes, osmagents, disintegrants, and lubricants. Exemplary excipients include microcrystalline cellulose; metallic salts of acids such as aluminum stearate, calcium stearate, magnesium stearate, sodium stearate, and zinc stearate; fatty acids, hydrocarbons and fatty alcohols such as stearic acid, palmitic acid, liquid paraffin, stearyl alcohol, and palmitol; fatty acid esters such as glyceryl (mono- and di-) stearates, triglycerides, glyceryl (palmitic stearic) ester, sorbitan monostearate, saccharose monostearate, saccharose monopalmitate, and sodium stearyl fumarate; alkyl sulfates such as sodium lauryl sulfate and magnesium lauryl sulfate; polymers such as polyethylene glycols, polyoxyethylene glycols, and polytetrafluoroethylene; and inorganic materials such as talc and dicalcium phosphate. Either animal or vegetable source magnesium stearate may be employed, however, the vegetable source material is generally preferred.

### The Water-Swellable Composition

The core of the controlled-release formulations of the present invention further comprises a water-swellable composition comprising a swelling agent. The water-swellable composition greatly expands as it imbibes water through the coating from the environment of use. As it expands, the water-swellable composition increases the pressure gradient within the core, causing extrusion of the fluidized drug-containing composition through the delivery port(s) into the environment of use. Normally, the water-swellable composition will have a swelling ratio of at least about 2, preferably about 3.5, and more preferably about 5. Preferably, the mass ratio of the drug-containing composition to the water-swellable composition has a value of at least 1.5.

The swelling agent present in the water-swellable composition typically comprises a water-swellable polymer that is generally present in an amount ranging from about 30 wt % to about 100 wt % of the water-swellable composition. Suitable swelling agents for the water-swellable composition are generally hydrophilic polymers that have swelling ratios of about 2.0 or greater. Exemplary hydrophilic polymers include polyoxomers such as PEO, cellulosics such as HPMC and HEC, and ionic polymers. A preferred class of swelling agents comprises the ionic polymers described above for use in the various embodiments of the drug-containing composition. Exemplary ionic polymeric swelling agents comprise sodium starch glycolate, sodium croscarmellose, polyacrylic acid, and sodium alginate. The water-swellable composition may further comprise osmotically effective agents, commonly referred to as “osmogens” or “osmagents.” The use of such osmotically effective agents in SCT controlled-release formulations is disclosed in detail in the aforementioned WO 01/47500. A generally preferred osmagent, useful in the water-swellable composition of the present invention, comprises finely powdered sodium chloride. The use of finely powdered sodium chloride reduces the potential for segregation in the layer comprising the water-swellable composition, and results in a less mottled appearance of the water-swellable composition aspect of the formulation.

In yet another preferred embodiment, the water-swellable composition of the core further comprises a tableting aid. The addition of a tableting aid to the water-swellable composition in an amount of between about 5 wt % and about 50 wt % results in a material that compresses to a preferred hardness for use in the SCT controlled-release formulations of the present invention. Generally, hydrophilic materials with good compression properties should be employed. Exemplary tableting aids include sugars such as lactose or xylitol, polymers such as microcrystalline cellulose, HPC, MC, or HPMC. Preferred tableting aids comprise microcrystalline cellulose, both standard grades and silicified versions.

It is generally desirable that the mixture of the swelling agent and tableting aid result in a material having a “strength” of at least 3 Kilopounds (Kp)/cm². Here, “strength” is the fracture force, also known as the core “hardness”, required to fracture a core formed from the material, divided by the maximum cross-sectional area of the core normal to that force. The ability to determine such “strength” is within the purview of one of ordinary skill in the art having benefit of the instant disclosure. Both the compressed water-swellable composition and resulting core should have a strength of at least 3 Kp/cm².

Where appropriate and/or desired, the water-swellable composition may further comprise solubility-enhancing agents or excipients that promote stability, tableting, or processing of the formulation. However, it is generally preferred that such excipients comprise a relatively minor portion of the water-swellable composition. In one preferred embodiment, the water-swellable composition contains a lubricant such as magnesium stearate. As disclosed hereinabove, vegetable source magnesium stearate is generally preferred. Pigments may also be included so as to provide sufficient contrast between the layers in the formulation corresponding to the drug-containing composition and the water-swellable composition. Generally preferred pigments comprise Blue Lake #2 and Red Lake #40.

### The Core

The core may comprise any conventionally shaped tablet that can be formed by an extrusion or compression process, and that can be subsequently coated and utilized for delivery of a drug to a mammal. In addition, the dosage form may comprise two or more relatively small tablets contained in a relatively large container, such as a capsule. The tablet can generally range in size from about 1 mm to about 10 cm for its longest dimension, however, the maximum size of the tablet will vary for different animal species. The core, following coating, can comprise the entire or a portion of the dosage form. The final dosage form can be for oral, rectal, vaginal, subcutaneous, or other known method of delivery into the environment of use. Exemplary core shapes are spheres, elipsoids, cylinders, capsule or caplet shapes, or any other known shape.

To prepare the formulation, the ingredients comprising the drug-containing composition and the water-swellable composition are first mixed or blended using processes known in the art. See for example, Lachman, et al., “The Theory and Practice of Industrial Pharmacy” (Lea & Febiger, 1986). For example, a portion of the ingredients of the drug-containing composition can first be blended, then wet granulated, dried, milled, and then blended with additional excipients prior to tableting. Similar processes can be used to form the water-swellable composition.

During granulation of the drug-containing composition, it has been observed that when PEO (Polyox WSR—N80, average molecular weight 200,000) is used in a high-shear granulation process, partial melting of the PEO can result, due to its relatively low melting point of 65-70° C. Therefore, when PEO is used in a high-shear granulation process, it is generally preferred to employ a lower impeller speed and/or granulate for shorter periods of time. It is also generally preferred to complete a dry blending step in a low-shear mixer, such as a V-blender or bin-blender prior to granulation. For blends comprising a PDE4D content of 5% or greater, uniform blends having suitable flow and compression characteristics are preferably obtained using a direct blend-mill-blend and compress process, i.e., without the need for granulation.

Once the component materials are properly mixed, the core is formed by using procedures well-known in the art. For example, to form cores in the form of tablets, the desired amount of drug-containing composition is placed in a tablet press and leveled by lightly tamping with the press. The desired amount of water-swellable composition is then added, and the tablet formed by compression. Alternatively, the water-swellable composition may be added to the tablet press first, followed by the drug-containing composition. The amount of force used to compress the tablet core will depend on the size of the dosage form, as well as the compressibility and flow characteristics of the compositions. Preferably, a pressure is used that results in a tablet having strength following tableting of at least 3 Kp/cm², more preferably a strength of between 3 and 20 Kp/cm².

During the process of manufacturing the formulation cores comprising PEO, it is frequently observed that there is some degree of flowing of the PEO between the space on the surfaces of the dies of the tablet tooling that results in a deleterious phenomenon called “crowning.” In extreme cases of crowning, the formed tablet core forms a raised edge on the tablet rim. Therefore, during the processing of such tablet cores, it is generally preferred that the compression force be lowered to an appropriate value so as to minimize crowning while maintaining adequate hardness and friability characteristics of the tablet. It is further preferred to employ new or polished tablet tooling. In general, if the compression force is reduced, the tablet core hardness is correspondingly reduced, and there is a greater possibility of increasing the friability of the formed tablet cores. It has been observed, however, that the friability of tablets comprising PEO is generally adequate for survival of the tablets during the tumbling process that occurs during the coating operation.

### THE COATING

Following formation of the core, the coating is applied. The coating should have both a sufficiently high water permeability that the drug can be delivered within the desired time frame, and high strength, while at the same time be easily manufactured. A water permeability value is chosen so as to control the rate at which water enters the core, thus controlling the rate at which drug is delivered to the environment of use. Where a high dose of the PDE4D inhibitor, or the pharmaceutically acceptable salt thereof, is required, the low solubility and high dose combine to make it preferable to employ a high permeability coating to achieve the desired drug release profile while keeping the tablet acceptably small in size. High strength is required to ensure the coating does not burst when the core swells as it imbibes water, leading to an uncontrolled delivery of the core contents to the use environment. The coating must be easily applied to the dosage form with high reproducibility and yield. Furthermore, the coating must be non-dissolving and non-eroding during release of the drug-containing composition, generally meaning that it be sufficiently water-insoluble so that drug is substantially entirely delivered through the delivery port(s), in contrast to delivery via permeation through coating.

As described hereinabove, the coating is highly water-permeable to allow rapid imbibition of water into the core and, as a result, rapid release of the drug-containing composition. A relative measure of the water permeability of the coating can be made by placing the prepared formulations in an open container which is, in turn, placed in an environmental chamber held at a constant temperature of 40° C., and a constant relative humidity of 75%. The initial rate of weight gain of the dry dosage forms, determined by plotting the weight of the dosage form versus time, divided by the surface area of the dosage form yields a value termed “water flux (40/75).” The water flux (40/75) for an individual formulation is known to be a useful relative measure of the water permeabilities of coatings. For the forms of a preferred embodiment of the present invention, in particular when a rapid release of the drug is desired, the coating has a water flux (40/75) of at least 1.0×10⁻³ gm/cm² hr.

Also as mentioned hereinabove, the coating should also have a high strength to ensure the coating does not burst when the core swells due to imbibition of water from the use environment. A relative measure of coating strength can be made by placing the tableted formulation into an aqueous medium for 10 to 24 hours, allowing the core to imbibe water, swell, and release drug to the media. The swollen formulation can then be tested in a hardness tester. The formulation is placed into the tester so that its delivery port(s) face(s) one side of the compression plates. The force, measured in Kp, required to rupture the coating is then quantified. The durability of the coating is then calculated by dividing the measured rupture force by the maximum cross-sectional area of the dosage form normal to the applied force. In a preferred embodiment of the invention, the coating should have a durability of at least 1 Kp/cm², preferably at least 2 Kp/cm², and most preferably at least 3 Kp/cm².

Coatings having such desirable characteristics comprise hydrophilic polymers such as plasticized and unplasticized cellulose esters, ethers, and ester-ethers. Particularly suitable polymers include CA, cellulose acetate butyrate, and ethyl cellulose. A particularly preferred class of polymers comprises cellulose acetates having acetyl contents of 25% to 42%. A preferred polymer is CA having an acetyl content of 39.8%, and specifically, CA 398-10 manufactured by Eastman Chemical Co.; Kingsport, Tenn., having an average molecular weight of about 40,000 daltons. Another preferred CA having an acetyl content of 39.8% is high molecular weight CA having an average molecular weight greater than about 45,000, and specifically, CA 398-30 (Eastman Chemical Co.) reported to have an average molecular weight of 50,000 daltons.

The coating process is conducted in conventional fashion by first forming a coating solution and then coating by dipping, fluidized bed coating, or preferably by pan coating. Accordingly, a coating solution is formed comprising the coating polymer and a solvent. Typical solvents useful with the cellulosic polymers noted hereinabove include acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, and mixtures thereof. A particularly preferred solvent is acetone. The coating solution typically will contain about 3 wt % to about 15 wt % of the polymer, preferably about 5 wt % to about 10 wt %, most preferably about 7 wt % to about 10 wt %.

The coating solution may further comprise pore-formers, non-solvents, or plasticizers in any amount so long as the polymer remains substantially soluble at the conditions used to form the coating, and as long as the coating remains water-permeable, and retains sufficient strength. Pore-formers and their use in fabricating coatings are described in U.S. Pat. Nos. 5,612,059 and 5,698,220, the disclosures of which are incorporated herein by reference in their entirety. Suitable pore-formers include polyethylene glycol (PEG), PVP, PEO, HEC, HPMC, and other aqueoussoluble cellulosics, water-soluble acrylate or methacrylate esters, polyacrylic acid and various copolymers and mixtures of these water soluble or water swellable polymers. Enteric polymers such as CAP and HPMCAS are also embraced within this class of polymers. A particularly preferred pore former is PEG having an average molecular weight from 1000 to 8000 daltons. A particularly preferred PEG is one having a molecular weight of 3350 daltons. When PEG is used as a pore former, the weight ratio of CA: PEG should range from about 6.5:3.5 to about 9:1.

The addition of a non-solvent to the coating solution is generally preferred. The phrase “non-solvent” denotes any material added to the coating solution that substantially dissolves in the coating solution, and reduces the solubility of the coating polymer, or polymers, in the solvent. In general, the function of the non-solvent is to impart porosity to the resulting coating. Porous coatings have a higher water permeability than an equivalent weight of a coating of the same composition that is not porous. The suitability and amount of a particular non-solvent candidate material can be evaluated by progressively adding the candidate non-solvent to the coating solution until it becomes cloudy. If this does not occur at any addition level up to about 50 wt % of the coating solution, it generally is not appropriate for use as a non-solvent. When clouding is observed, termed the “cloud point”, an appropriate level of non-solvent for maximum porosity is the amount just below the cloud point. If lower porosities are desired, the amount of non-solvent can be reduced as low as desired. Suitable coatings can be obtained when the concentration of non-solvent in the coating solution is greater than about 20% of the non-solvent concentration that results in the cloud point.

Suitable non-solvents comprise materials that have appreciable solubility in the solvent, and that lower the coating polymer solubility in the solvent. The preferred non-solvent depends on the solvent and the coating polymer chosen. In the case of using a volatile polar coating solvent, such as acetone or methyl ethyl ketone, suitable non-solvents include water, glycerol, ethylene glycol and its low molecular-weight oligomers (e.g., less than about 1,000 daltons), propylene glycol and its low molecular weight oligomers (e.g., less than about 1,000 daltons), C₁-C₄ alcohols such as methanol or ethanol, ethylacetate, acetonitrile, and the like.

In general, to maximize pore formation, the non-solvent should have similar or less volatility than the coating solution solvent such that, during initial evaporation of the solvent during the coating process, sufficient non-solvent remains to cause phase separation to occur. In many cases, where a coating solution solvent such as acetone is used, water is a suitable non-solvent. For acetone solutions comprising 7 wt % CA and 3 wt % PEG, the cloud point at ambient temperature is at about 23 wt % water. Thus, the porosity and, in turn, the water permeability can be controlled by varying the water concentration up to near the cloud point. For acetone solutions comprising CA and PEG with a total concentration of about 10 wt %, it is desired that the coating solution contain at least 4 wt % water to obtain a suitable coating. When a higher porosity, and thus a higher water permeability is desired, the coating solution should contain at least about 15 wt % water.

When using CA 398-10, exemplary coating solution weight ratios of CA:PEG 3350:water are 7:3:5, 8:2:5, and 9:1:5, with the remainder of the solution comprising a solvent such as acetone. Thus, for example, in a solution having a weight ratio of CA:PEG 3350:water of 7:3:5, CA comprises 7 wt % of the solution, PEG 3350 comprises 3 wt % of the solution, water comprises 5 wt % of the solution, and acetone comprises the remaining 85 wt %.

Preferred coatings are generally porous even in the dry state (prior to delivery to the aqueous environment of use). The term “porous” means that the coating has a dry-state density less than the density of the nonporous coating material. The phrase “nonporous coating material” denotes a coating material formed by using a coating solution containing no non-solvent, or the minimum amount of non-solvent required to produce a homogeneous coating solution. The coating in the dry state has a density that is less than 0.9 times, and more preferably less than 0.75 times that of the nonporous coating material.

The coatings may also be asymmetric, meaning that there is a gradient of density throughout the coating thickness. Generally, the outside surface of the coating will have a higher density than the coating nearest the core.

The coating can optionally comprise a plasticizer. A plasticizer generally swells the coating polymer such that the polymer's glass transition temperature is lowered, its flexibility and toughness are increased, and its permeability altered. When the plasticizer is hydrophilic, such as polyethylene glycol, the water permeability of the coating is generally increased. When the plasticizer is hydrophobic, such as diethyl phthalate or dibutyl sebacate, the water permeability of the coating is generally decreased.

The weight of the coating around the core depends on the composition and porosity of the coating, the surface to volume ratio of the formulation, and the desired drug release rate, but generally should be present in an amount ranging from about 3 wt % to 30 wt %, preferably from about 8 wt % to 25 wt %, based on the weight of the uncoated core. However, a coating weight of at least about 8 wt % is generally preferred so as to assure sufficient strength for reliable performance, and more preferably a coating greater than about 13 wt %.

While porous coatings based on CA, PEG, and water yield excellent results, other pharmaceutically acceptable materials may be used so long as the coating has the requisite combination of high water permeability, high strength, and ease of manufacture. Further, such coatings may be dense, or asymmetric, having one or more dense layers and one or more porous layers, as described in U.S. Pat. Nos. 5,612,059 and 5,698,220, the disclosures of which are incorporated herein by reference in their entirety.

The coating also comprises at least one delivery port in communication with both the interior and exterior of the coating to permit release of the drug-containing composition to the exterior of the formulation. The number of delivery ports may vary from at least 1 to about 10, or more. At least one delivery port should be formed on the side of the coating that is adjacent to the drug-containing composition, so that such composition will be extruded out of the port by the swelling action of the water-swellable composition. The delivery port(s) may range in size from about the size of the drug particles, i.e., about 1 micron to about 100 microns in diameter, or may be termed pores up to about 5,000 microns in diameter. The shape of such delivery port(s) many be substantially circular, in the form of a slit, or other conventional shape. The delivery port(s) may be formed by post-coating mechanical or thermal means, or with a beam of light (e.g., a laser), a beam of particles, or other high-energy source, or may be formed in situ by rupture of a small portion of the coating. The delivery port(s) may also be formed in situ by the erosion of a plug of water-soluble material, or by rupture of a thinner portion of the coating over an indentation in the core. Additionally, the delivery port(s) may comprise a large number of holes or pores formed during the coating process, as disclosed in U.S. Pat. Nos. 5,612,059 and 5,698,220, the disclosures of which are incorporated herein by reference in their entirety. In aggregate, the total surface area of core exposed by the delivery ports is less than about 5%, and more typically less than about 1%.

Especially preferred are SCT formulations with one delivery port as these are more likely to provide a time lag before initiation of delivery as well as extended release of the PDE4D inhibitor or pharmaceutically acceptable salt thereof.

The invention further provides methods of treating disorders and conditions mediated by the PDE4D isozyme, which comprise administering to a mammal in need of such treatment a therapeutically effective amount of a PDE4D inhibitor, or a pharmaceutically acceptable salt thereof, in a controlled-release formulation of the present invention. Preferably, the PDE4 inhibitor comprises the compound (R)-2-[4-({[2-(benzo[1,3]dioxol-5-yloxy)-pyridine-3-carbonyl]-amino}-methyl)-3-fluoro-phenoxy]-propionic acid, or a pharmaceutically acceptable salt thereof, or the compound 2-(4-fluorophenoxy)-N-[4-(1-hydroxy-1-methyl-ethyl)-benzyl]-nicotinamide, or a pharmaceutically acceptable salt thereof.

Preferred disorders and conditions treatable according to the present methods are selected from the group consisting of:

(a) inflammatory diseases and conditions selected from the group consisting of joint inflammation, rheumatoid arthritis, rheumatoid spondylitis, osteoarthritis, inflammatory bowel disease, ulcerative colitis, chronic glomerulonephritis, dermatitis, and Crohn's Disease;

(b) respiratory diseases and conditions selected from the group consisting of asthma, acute respiratory distress syndrome, chronic obstructive pulmonary disease, bronchitis, chronic obstructive airway disease, and silicosis;

(c) infectious diseases and conditions selected from the group consisting of sepsis, septic shock, endotoxic shock, gram negative sepsis, toxic shock syndrome, fever and myalgias due to bacterial, viral or fungal infection, and influenza;

(d) immune diseases and conditions selected from the group consisting of autoimmune diabetes, systemic lupus erythematosis, graft v. host reaction, allograft rejections, multiple sclerosis, psoriasis, and allergic rhinitis; and

(e) other diseases and conditions selected from the group consisting of bone resorption diseases, reperfusion injury, cachexia secondary to infection or malignancy, cachexia secondary to human immunodeficiency syndrome (AIDS), human immunodeficiency virus (HIV), infection, or AIDS related complex (ARC), keloid formation, scar tissue formation, Type 1 diabetes mellitus, and leukemia.

Especially preferred disorders and conditions treatable according to the present methods are asthma, acute respiratory distress syndrome, chronic obstructive pulmonary disease, bronchitis, chronic obstructive airway disease, and silicosis.

Typically, dosages of PDE4D inhibitors, or the pharmaceutically acceptable salts thereof, comprising the instant controlled-release formulations range from about 0.1 μg to about 50.0 mg/kg of body mass per day, preferably from about 5.0 μg to about 5.0 mg/kg of body mass per day, more preferably from about 10.0 μg to about 1.0 mg/kg of body mass per day, and, most preferably, from about 20.0 μg/kg to about 0.5 mg/kg of body mass per day. Some variability, however, some variability in the general dosage ranges may be required depending upon the age and weight of the patient being treated, the particular PDE4D inhibitor being administered, the nature and kind of concurrent therapy, if any, the frequency of treatment and the nature of the effect desired, and the like. The determination of dosage ranges and optimal dosages for a particular patient is well within the ability of one of ordinary skill in the art having the benefit of the instant disclosure.

The invention further provides methods of reducing PDE4D inhibitor treatment-induced nausea and/or emesis in a mammal which comprise administering the PDE4D inhibitor, or a pharmaceutically acceptable salt thereof, to the mammal in the form of a controlled-release formulation of the present invention. Preferred PDE4 inhibitors useful in such methods comprise the compounds of structural formulae (I) and (Ia) hereinbelow, and the pharmaceutically acceptable salts thereof.

The invention further provides controlled-release pharmaceutical formulations having a Tₘₐₓ of greater than about 1.5 hours. A Tₘₐₓ of between about three hours and six hours is generally preferred; a Tₘₐₓ of between about 12 hours and 18 hours is especially preferred. As employed herein, the terms Cₘₐₓ and Tₘₐₓ are well-known pharmacokinetic parameters obtained from the plasma concentration vs. time profiles.

The invention further provides controlled-release formulations wherein less than about 80% of the PDE4D inhibitor, or the pharmaceutically acceptable salt thereof, is released in vivo at about 1.5 hours. A release of greater than about 80% of the PDE4D inhibitor, or the pharmaceutically acceptable salt thereof, in vivo between about three hours and six hours is generally preferred; a release of greater than about 80% of the PDE4D inhibitor, or the pharmaceutically acceptable salt thereof, in vivo between about 12 hours and 18 hours is especially preferred.

The invention further provides controlled-release formulations wherein less than about 80% of the PDE4D inhibitor, or the pharmaceutically acceptable salt thereof, is released in vitro at about 1.5 hours.

The invention further provides controlled-release formulations that have an in vivo delivery time lag prior to initiation of release of the PDE4D inhibitor, or the pharmaceutically acceptable salt thereof, of between about 0.5 hours and about four hours. An in vivo lag time of between about 0.5 hours and about two hours is generally preferred.

The invention further provides controlled-release formulations that have an in vitro delivery time lag prior to initiation of release of the PDE4D inhibitor, or the pharmaceutically acceptable salt thereof, of between about 0.5 hours and about four hours. An in vitro lag time of between about 0.5 hours and about two hours is generally preferred.

The invention further provides SCT controlled-release formulations of compound (I) having a Cₘₐₓ to Tₘₐₓ ratio of less than about 40. An SCT formulation of compound (I) having a Cₘₐₓ to Tₘₐₓ ratio of between about 15 and about 40 is generally preferred; a Cₘₐₓ to Tₘₐₓ ratio of between about 15 and about 20 is especially preferred.

Although any PDE4D inhibitor, or pharmaceutically acceptable salt thereof, may be employed in the controlled-release formulations and methods of the present invention, generally preferred PDE4D inhibitors comprise the compounds (R)-2-[4-({[2-(benzo[1,3]dioxol-5-yloxy)-pyridine-3-carbonyl]-amino}-methyl)-3-fluoro-phenoxy]-propionic acid, and 2-(4-fluorophenoxy)-N-[4-(1-hydroxy-1-methyl-ethyl)-benzyl]nicotinamide, i.e., the compounds of structural formulae (I) and (Ia) respectively hereinbelow, and the pharmaceutically acceptable salts thereof.

The preferred PDE4D inhibitor of structural formula (I), and the pharmaceutically acceptable salts thereof, may be prepared according to conventional preparative methods known to one of ordinary skill in the art or, more conveniently, as outlined hereinbelow in Scheme 1. The additionally preferred PDE4D inhibitor of structural formula (Ia) may be prepared as disclosed in commonly assigned U.S. Pat. No. 6,380,218, the disclosure of which is incorporated herein by reference in its entirety.

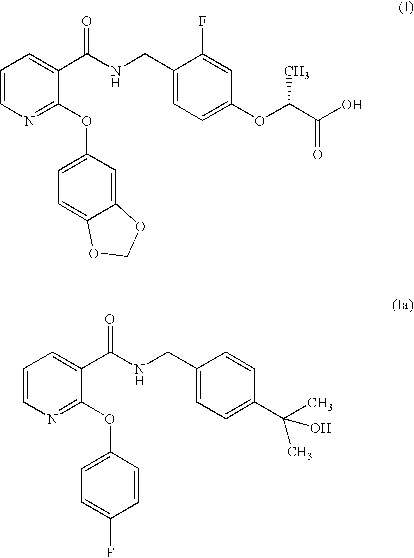

A preferred method for preparing the compound of structural formula (I) is illustrated hereinbelow in Scheme 1. Preferred methods for preparing the nicotinic acid (III) and amine intermediates (IV) are depicted hereinbelow in Schemes 2 and 3 respectively.

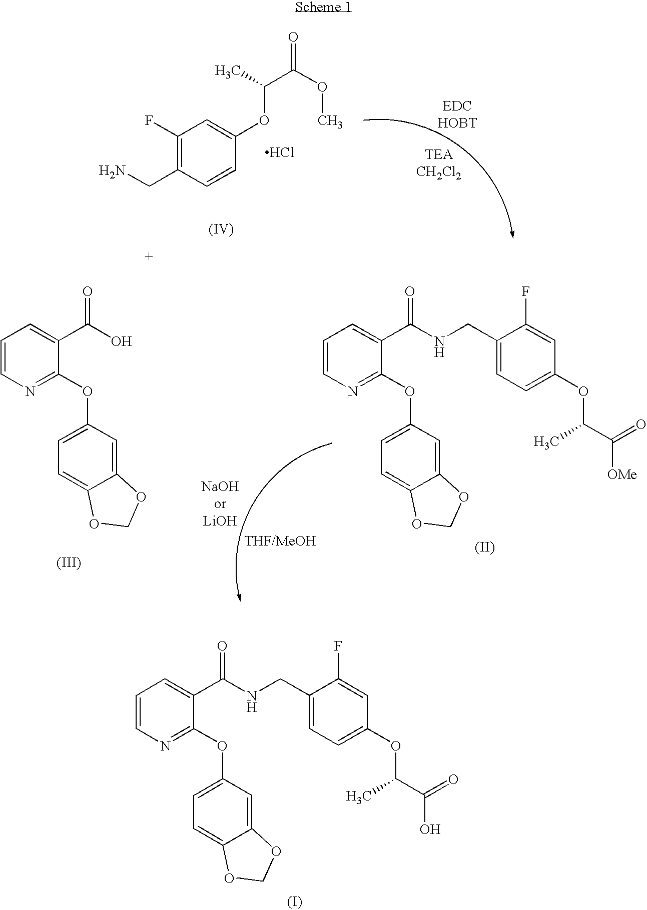

In Scheme I hereinabove, 2-(benzo-[1,3]dioxolo-5-yloxy)-nicotinic acid (III) is condensed with (R)-2-(4-aminomethyl-3-fluoro-phenoxy)-propionic acid methyl ester hydrochloride (IV), in the presence of a coupling agent, such as 1,1′-carbonyldiimidazole (CDI), 1-(3-dimethylaminopropyl)-3-ethylcarbodiimide (EDC), or 1,3-dicyclohexylcarbodiimide (DCC), and an organic base, such as triethylamine. Such condensation is typically effected in an aprotic solvent, such as N,N-dimethylformamide, or dichloromethane, preferably at ambient temperature. The methyl ester (II) so formed is then saponified with aqueous base, for example, lithium hydroxide or sodium hydroxide, in a protic solvent, preferably methanol, or a mixture or tetrahydrofuran/methanol, to afford the compound of formula (I).

The pharmaceutically acceptable salts of the compound of formula (I), preferably the basic addition salts, may be prepared according to conventional methods. For example, the preferred basic addition salts may be prepared by contacting compound (I) with a stoichiometric amount of an appropriate organic or inorganic base to provide the corresponding basic addition salt. Inorganic basic addition salts of the present invention include, but are not limited to, aluminum, ammonium, calcium, copper, ferric, ferrous, lithium, magnesium, manganic, manganous, potassium, sodium, and zinc salts. Preferred among the recited inorganic basic addition salts are ammonium; the alkali metal salts sodium and potassium; and the alkaline earth metal salts calcium and magnesium. Salts of the compound of formula (I) derived from non-toxic organic bases include, but are not limited to, salts of primary, secondary, and tertiary amines, substituted amines including naturally-occurring substituted amines, cyclic amines, and basic ion exchange resins, e.g., arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine, and tris(hydroxymethyl)-methylamine (tromethamine).

The pharmaceutically acceptable salts of the compound of formula (Ia), preferably the acid addition salts, may also be prepared according to conventional methods. For example, the preferred acid addition salts may be prepared by contacting compound (Ia) with a stoichiometric amount of an appropriate inorganic or organic acid to provide the corresponding acid addition salt. Inorganic acid addition salts may comprise, for example, the hydrochloric, hydrobromic, nitric, sulfuric, and phosphate addition salts. Organic acid addition salts may comprise, for example, the acetate, besylate, citrate, fumarate, tartrate, and tosylate addition salts.

A preferred method for preparing the nicotinic acid intermediate (III) is illustrated hereinbelow in Scheme 2.

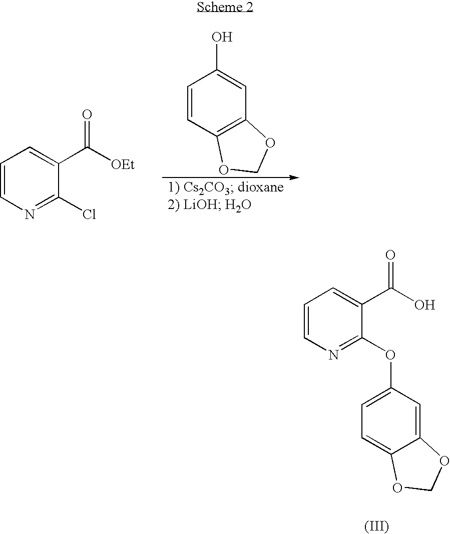

In Scheme 2, Step 1, hereinabove, 2-chloro-nicotinic acid ethyl ester is condensed with benzo[1,3]dioxol-5-ol (sesamol) in the presence of an inorganic base, such as potassium carbonate or cesium carbonate. Such condensation is typically effected in an aprotic solvent, such as dimethylformamide, tetrahydrofuran, or dioxane at elevated temperature. Preferably, the condensation is effected in the presence of cesium carbonate in refluxing dioxane. In Scheme 2, Step 2, the nicotinic acid intermediate (III) is most conveniently prepared by in situ hydrolysis of the resulting ethyl ester precursor of (III) resulting from the condensation of 2-chloro-nicotinic acid ethyl ester and benzo[1,3]dioxol-5-ol. However, one of ordinary skill in the art will appreciate that such ethyl ester precursor of (III) may, if desired and/or appropriate, be isolated and hydrolyzed in a separate step.

A preferred method for preparing the amine intermediate (IV) is illustrated hereinbelow in Scheme 3.

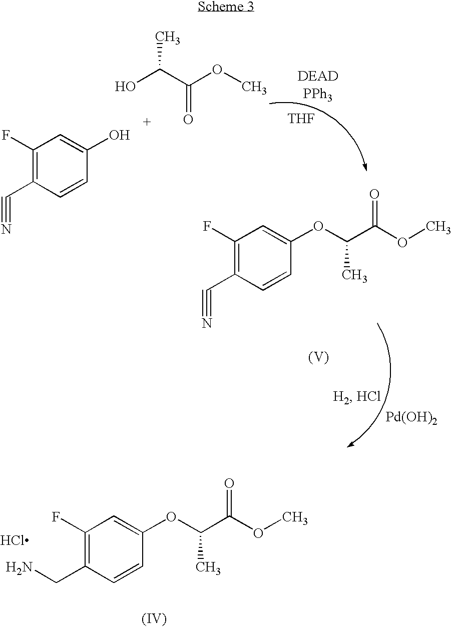

In Scheme 3 hereinabove, 2-fluoro-4-hydroxybenzonitrile is condensed with methyl (S)-(−)-lactate via the so-called Mitsunobu reaction to afford benzonitrile (V). Such condensation is typically effected in the presence of a dehydrating reagent, for example, a stoichiometric amount of a diazocarboxyl compound, such as diethyl azodicarboxylate, and a phosphine, for example, triphenylphosphine. The reaction is effected in a reaction-inert, aprotic solvent, such as tetrahydrofuran. The functionalized benzonitrile (V) so formed is then reduced, preferably by catalytic hydrogenation with palladium hydroxide in a protic solvent, such as methanol. The resulting free amine is most conveniently isolated as the acid addition salt thereof. A preferred acid addition salt comprises the hydrochloride salt (IV). The acid addition salts, including the preferred hydrochloride addition salt (IV), may be prepared according to known methods. The preferred hydrochloride addition salt (IV) is preferably prepared by performing the catalytic hydrogenation of benzonitrile (V) in the presence of at least one molar equivalent of hydrochloric acid.

### Experimental

Chemical Syntheses

With reference to the synthetic outlines depicted in Schemes 1, 2, and 3 hereinabove, compound (I) is prepared utilizing the intermediates of the following Examples. Other synthetic variations will be known, or apparent in light of the instant disclosure, to one of ordinary skill in the art. Unless otherwise noted, all reactants were obtained commercially.

### EXAMPLE 1

### 2-(Benzo-[1,3]dioxolo-5-yloxy)-nicotinic Acid (III)

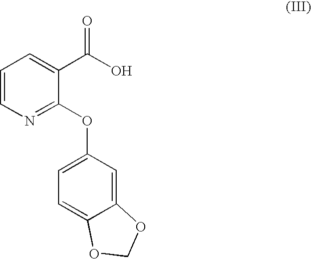

2-Chloro-nicotinic acid ethyl ester (10 g), benzo[1,3]dioxol-5-ol (sesamol, 8.2 g), and cesium carbonate (21 g) were mixed in anhydrous dioxane (40 mL) and the resulting slurry was heated to reflux for 16 hours. In a separate flask, lithium hydroxide (12.9 g) was dissolved in water (80 mL) with warming and then added to the refluxing mixture, which was heated for an additional four hours. The mixture was cooled to ambient temperature and then concentrated in vacuo to remove the dioxane. Concentrated hydrochloric acid was added dropwise until the pH=3. The acidified solution was then extracted with ethyl acetate (7×100 mL) to yield the crude product, which was recrystallized from ethyl acetate to yield the purified title compound (10.8 g).

¹H NMR (CD₃OD): δ 8.28 (dd, J=8 and 2 Hz, 2H), 7.13 (m, 1H), 6.79 (d, J=8 Hz, 1H), 6.62 (s, J=2 Hz, 1H), 6.53 (dd, J=8 and 2 Hz, 1H), 5.95 (s, 2H).

### EXAMPLE 2

### (R)-2-(4-Cyano-3-fluoro-phenoxy)-propionic Acid Methyl Ester (V)

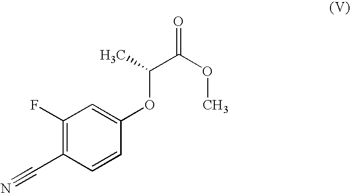

To a stirred solution of 2-fluoro-4-hydroxybenzonitrile (0.2 g, 1.5 mmol), methyl (S)-(−)-lactate (0.14 mL, 1.5 mmol) and triphenylphosphine (1.15 g, 4.4 mmol) in tetrahydrofuran at room temperature, diethyl azodicarboxylate (0.67 mL, 4.4 mmol) was added dropwise. The mixture was stirred at room temperature overnight, diluted with ethyl acetate and washed successively with dilute aqueous sodium hydroxide, dilute aqueous hydrochloric acid, brine, and dried over sodium sulfate. The solvents were then stripped off in vacuo. The resulting oil was washed with diethyl ether and the precipitate was filtered off. The mother liquor was adsorbed onto silica gel and then product purified by flash column chromatography (20% dichloromethane/hexanes), affording 0.12 g of a pink oil (36% yield).

¹H NMR (CDCl₃): δ7.51 (t, J=7.5 Hz, 1H), 6.71 (d, J=9 Hz, 1H), 6.67 (d, J=10 Hz, 1H), 4.78 (q, J=7 Hz, 1H), 3.77 (s, 3H), 1.64 (d, J=7 Hz, 3H).

### EXAMPLE 3

### (R)-2-(4-Aminomethyl-3-fluoro-phenoxy)-propionic Acid Methyl Ester Hydrochloride

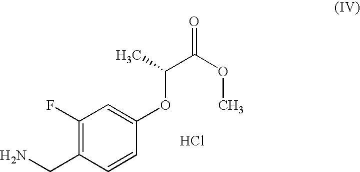

The title compound of Example 2 (6.5 g, 29 mmol) and palladium hydroxide (900 mg) were combined with 2.5 mL of concentrated hydrochloric acid in 200 mL of methanol and hydrogenated for 18 hours. The mixture was filtered through diatomaceous earth, concentrated by azeotropic distillation with ethanol (1×100 mL), and concentrated in vacuo to a solid. The product was suspended in 100 mL of diethyl ether, filtered, and dried to afford 7.5 g (98% yield) of the title compound.

¹H NMR (CDCl₃): δ 7.41 (t, J=8 Hz, 1H), 6.90 (br, 2H), 6.58 (m, 2H), 4.69 (q, J=7 Hz, 1H), 4.00 (s, 2H), 3.71 (s, 3H), 1.56 (d, J=7 Hz, 3H).

### EXAMPLE 4

### (R)-2-[4-({[2-(Benzo[1,3]dioxol-5-yloxy)-pyridine-3-carbonyl]-amino}-methyl)-3-fluoro-phenoxy]-propionic Acid Methyl Ester (II)

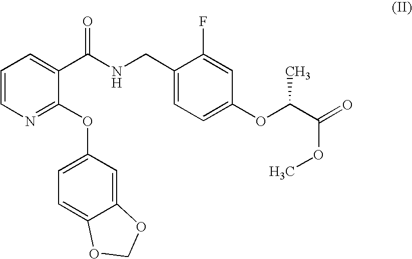

The title compound of Example 1 (221.9 g, 0.857 mol), the title compound of Example 3 (226.0 g, 0.857 mol), 1-hydroxybenzotriazole (127.3 g, 0.943 mol), N-ethyl-N′-(3-dimethylaminopropyl)carbodiimide hydrochloride (181.0 g, 0.943 mol), and triethylamine (119.2 mL, 86.6 g, 0.857 mol) were combined in 14 L of dichloromethane, and the resulting mixture was stirred at room temperature overnight. The mixture was washed with water (3×4 L), filtered through diatomaceous earth, and treated with decolorizing charcoal. The mixture was dried over magnesium sulfate, filtered through diatomaceous earth, and concentrated in vacuo to a solid. The solid was suspended in 2.5 L of diethyl ether and stirred overnight at ambient temperature. The solid was collected by filtration to furnish 347.3 g (87% yield) of the title compound.

MS (M/Z): 469 (M⁺+1, 20), 455 (M⁺−14, 100).

### EXAMPLE 5

### (R)-2-[4-({[2-(Benzo[1,3]dioxol-5-yloxy)-pyridine-3-carbonyl]-amino}-methyl)-3-fluoro-phenoxy]-propionic Acid (I)

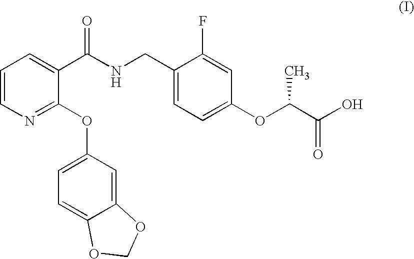

The title compound of Example 4 (344.0 g, 0.734 mol) was combined slowly with 1N sodium hydroxide (1.47 L), followed by methanol (5.18 L) with ice-bath cooling. The mixture was stirred at room temperature overnight and then diluted with 3 L of water. The mixture was cooled in an ice-bath and 735 mL of 2N hydrochloric acid was added slowly dropwise. The solid was collected, dissolved in 7 L of dichloromethane, and the solution washed with brine (1×2 L). The solution was dried over sodium sulfate, filtered through diatomaceous earth, and concentrated in vacuo to a furnish solid which was recrystallized from 2.5 L of acetontrile. There was obtained 276 g of crude product. The solid was pulped in a mixture of 2.76 L hexanes/830 mL ethyl acetate/140 mL methanol, and refluxed for 30 minutes. Upon cooling, the solid was collected, washed with hexanes, and dried to afford 253.6 g (76% yield) of the title compound, m.p. 151-152.5° C.

Anal. Calc'd. for C₂₃H₁₉FN₂O₇: C, 60.79; H, 4.21, N, 6.16. Found: C, 60.86; H, 4.35, N, 6.15.

¹H NMR (CDCl₃): δ 8.59 (dd, J=2 and 8 Hz, 1H), 8.31 (t, J=6 Hz, 1H), 8.21 (dd, J=2 and 5 Hz, 1H), 7.30 (t, J=8 Hz, 1H), 7.12 (dd, J=5 and 8 Hz, 1H), 6.81 (d, J=8 Hz, 1H), 6.61 (m, 3H), 6.00 (s, 2H), 4.74 (q, J=7 Hz, 1H), 4.63 (d, J=6 Hz, 2H), 1.64 (d, J=7 Hz, 3H).

Pharmaceutical Formulations

The controlled-release formulations of the present invention may be prepared according to the generalized methodologies disclosed in the aforementioned WO 01/47500. Specific preparations of such formulations, including certain preferred embodiments thereof, are set forth in detail in the following Examples.

### EXAMPLE 1a

One and 10 mg dosage tablets of the formulations of the present invention comprising (R)-2-[4-({[2-(benzo[1,3]dioxol-5-yloxy)-pyridine-3-carbonyl]-amino}-methyl)-3-fluoro-phenoxy]-propionic acid (I) were prepared as follows.

Drug-Containing Composition

A blend of (R)-2-[4-({[2-(benzo[1,3]dioxol-5-yloxy)-pyridine-3-carbonyl]-amino}-methyl)-3-fluoro-phenoxy]-propionic acid (I), polyethylene oxide (Polyox WSR N80; Dow Chemical Co.; Midland, Mich.), lactose Fast-Flo 316 (Foremost Farms USA; Baraboo, Wis.), and hydroxypropyl cellulose (Klucel E F; Aqualon; Wilmington, Del.) was mixed in a V-blender (Patterson-Kelly; East Stroudsburg, Pa.) for 30 minutes until a homogenous blend was achieved. The blend was then granulated in an SP1 high shear mixer (Niro, Aeromatic Div.; Columbia, Md.), wet milled, and dried overnight in a tray mixer. The dried granulation was milled in a Fitzpatrick M5A mill (Fitzpatrick; Elmhurst, Ill.), and lubricated with magnesium stearate in a V-blender.

Water-Swellable Composition

The water-swellable composition was produced by combining polyethylene oxide (Polyox WSR Coagulant Grade), sodium chloride powder, and Red Lake #40 pigment (Sensient Pharmaceutical Technologies; South Plainfield, N.J.). The mixture was dry blended in a V-blender to achieve a uniform blend.

The formulations for the drug-containing compositions and water-swellable compositions for the 1 mg dosage formulations of compound (I) are provided in Tables 1 and 2 respectively. The formulations for the 10 mg dosage formulations of compound (I), prepared in a manner similar to that for the 1 mg dosages, are provided in Tables 3 and 4.

TABLE 1
1 mg Tablet
Drug-Containing Composition (layer weight 100 mg)

| | | | Batch | |
|---|---|---|---|---|
| # | Ingredient | Purpose | (%) | mg/tab |
| 1 | Compound (I) | PDE4D Inhibitor | 1.000% | 1.00 |
| 2 | PEO WSR N80 | Entraining Agent | 66.000% | 66.00 |
| 3 | Lactose Fast-Flo | Diluent | 27.000% | 27.00 |
| 4 | HPC (Klucel EF) | Binder | 5.000% | 5.00 |
| 5 | Magnesium | Lubricant | 1.000% | 1.00 |
| | Stearate | | | |
| | | Total | 100.000% | 100.000 |

TABLE 2
1 mg Tablet
Water-Swellable Composition (layer weight 50 mg)

| # | Ingredient | Purpose | Batch (%) | mg/tab |
|---|---|---|---|---|
| 1 | Polyox WSR | Swelling | 65.000% | 32.50 |
| | Coagulant Grade | Agent | | |
| 2 | Sodium chloride | Osmagent | 34.400% | 17.20 |
| 3 | Red Lake #40 | Pigment | 0.100% | 0.05 |
| 4 | Magnesium Stearate | Lubricant | 0.500% | 0.25 |
| | | Total | 100.000% | 50.000 |

TABLE 3
10 mg Tablet
Drug-Containing Composition (layer weight 200 mg)

| | | | Batch | |
|---|---|---|---|---|
| # | Ingredient | Purpose | (%) | mg/tab |
| 1 | Compound (I) | PDE4D Inhibitor | 5.000% | 10.00 |
| 2 | PEO WSR N80 | Entraining Agent | 66.000% | 132.00 |
| 3 | Lactose Fast-Flo | Diluent | 23.000% | 46.00 |
| 4 | HPC (Klucel EF) | Binder | 5.000% | 10.00 |
| 5 | Magnesium Stearate | Lubricant | 1.000% | 2.00 |
| | | Total | 100.000% | 200.000 |

TABLE 4
10 mg Tablet
Water-Swellable Composition (layer weight 100 mg)

| # | Ingredient | Purpose | Batch (%) | mg/tab |
|---|---|---|---|---|
| 1 | Polyox WSR | Swelling | 65.000% | 65.00 |
| | Coagulant Grade | Agent | | |
| 2 | Sodium chloride | Osmagent | 34.400% | 34.40 |
| 3 | Red Lake #40 | Pigment | 0.100% | 0.10 |
| 4 | Magnesium Stearate | Lubricant | 0.500% | 0.50 |
| | | Total | 100.000% | 100.000 |

Core

The drug-containing composition and the water-swellable composition were compressed into bilayer tablet cores on a Hata multiplayer tablet press (Elizabeth Hata; North Huntingdon, Pa.) by compressing 200 mg of the drug-containing composition and 100 mg of the water-swellable composition for the 10 mg formulation of compound (I) using 11/32″ Standard Round Concave (SRC) plain faced tooling. The 1 mg formulation of compound (I) was prepared by compressing 100 mg of the drug-containing composition and 50 mg of the water-swellable composition using 9/32″ SRC tooling. The 1 mg tablet core weighed 150 mg, and the 10 mg tablet weighed 300 mg.

Coating

The tablet cores were coated in a Vector LDCS-20 coating pan (Vector Corp.; Marion, Iowa). The coating level was 30% w/w (45 mg) for the 150 mg (1 mg) tablet, and 20% w/w (60 mg) for the 300 mg (10 mg) tablet. The coating components are indicated in Table 5, and process conditions are listed hereinbelow Table 6. The coated tablets were placed in a tray drier at 45° C. for 24 hours to remove any residual coating solvents.

TABLE 5
Coating Formulation

| # | Ingredient | Batch (%) |
|---|---|---|
| 1 | Purified Water | 5.000% |
| 2 | PEG 3350 | 2.000% |
| 3 | Acetone | 85.000% |
| 4 | Cellulose Acetate (398-10) | 8.000% |
| | | 100.000% |

TABLE 6
Coating Process in LDCS-20 Coating Pan

| Process Parameter | Setting |
|---|---|
| Equipment | LDCS-20 coating pan |
| Batch Size | 600-1000 g |
| Pan Speed | 20 rpm |
| Process Air Flow | 30-40 cfm |
| Inlet Air Temperature | 43° C. |
| Outlet Air Temperature | 30° C. |
| Atomization/Pattern Air | 15 psi |
| Spray Rate | 20 g/min |

Delivery Port

A 0.9 mm diameter delivery port was laser-drilled through the coating on the drug-containing composition side of the tablet using either a laser-drilling unit, or a drill press having a 0.9 mm drill bit. Process parameters for the laser-drilling unit are provided hereinbelow Table 7.

TABLE 7
Compound (I) Formulation
Laser Drilling Parameters

| Process Parameter | Setting |
|---|---|
| Mask | 7.5 mm |
| Mask Position | 540 mm |
| Demagnification | 8.33 |
| HV level | 5 |
| Internal Trigger | 15 Hz |
| Shot Repeat | 14 |
| Total Shots | 15 |

In Vitro Performance of Compound (I) Formulations

A salient attribute of the SCT controlled-release formulations of the invention is the delivery of the PDE4D inhibitor, or the pharmaceutically acceptable salt thereof, to an environment of use in a controlled manner. In vitro dissolution tests, which will be well-known to one of ordinary skill in the art, may be employed to determine whether a dosage formulation provides a controlled-release profile. One example of such a dissolution test is disclosed hereinbelow. Additional examples of such dissolution tests are disclosed in the aforementioned WO 01/47500.

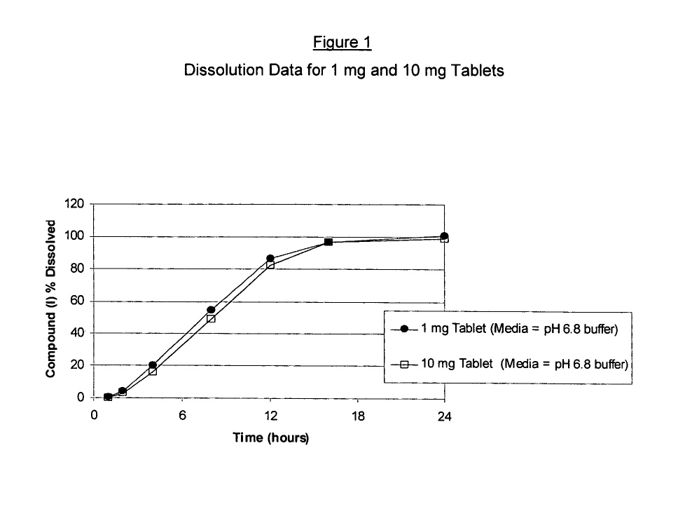

Dissolution of the 1 mg and 10 mg SCT controlled-release formulations comprising compound (I) was determined using an USP Apparatus II (Hansen Research Corp.; Chatsworth, Ga. or Distek Inc.; North Brunswick, N.J.) (rotating paddles at 50 rpm in 500 mL of 10 mM K₂PO₄ buffer for the 1 mg formulation, or 900 of 10 mM K₂PO₄ buffer for the 10 mg formulation). The amount of compound (I) dissolved was determined by reversed-phase HPLC. The dissolution profile exhibited about a two-hour time lag, followed by near zero order release with about 80% of compound (I) dissolved in 12 hours. FIG. 1 shows the average release rate of the 1 and 10 mg formulations.

[image]

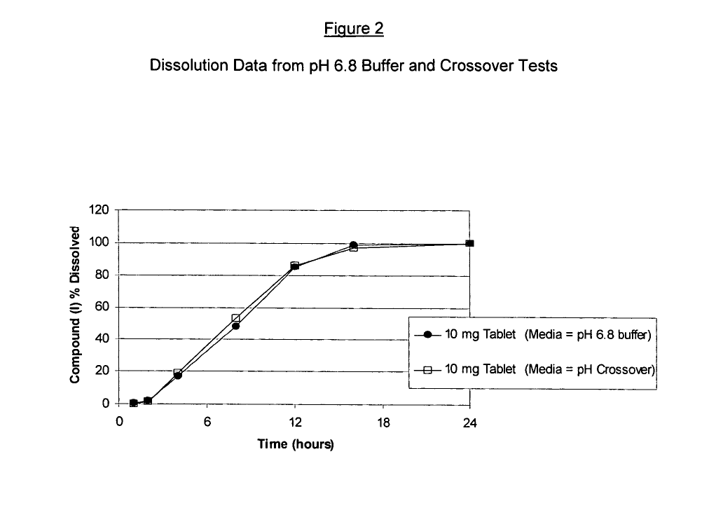

FIG. 2 shows data from a pH-crossover test. The pH-crossover test was employed to simulate in vivo (physiological) exposure conditions during passage of the instant SCT formulations from the stomach through the small intestine. In the pH-crossover test, tablets were placed in pH 1.2 simulated gastric fluid without enzymes for two hours, and then transferred to pH 6.8 phosphate buffer. The results indicate no significant change in the release profile between straight pH 6.8 buffer and the pH-crossover dissolution test.

[image]

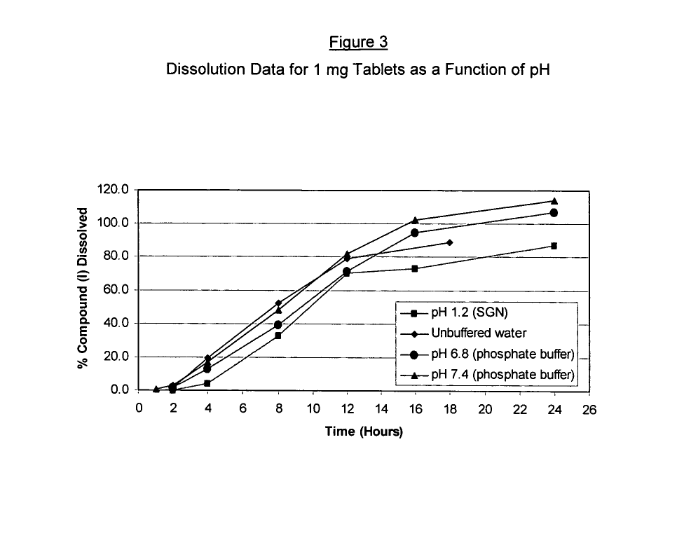

FIG. 3 shows dissolution data for the 1 mg controlled release of compound (I) as a function of dissolution media pH. The in vitro dissolution of compound (I) from the 1 mg formulation was essentially independent of the pH of the dissolution media. In order to determine the amount of compound (I) dissolved in simulated gastric fluid at pH 1.2, the amount remaining in the formulations was assayed and subtracted from the amount initially present. This procedure was employed because, at the lower pH, the solubility of compound (I) is lower and sink conditions cannot be maintained during dissolution.

[image]

[image]

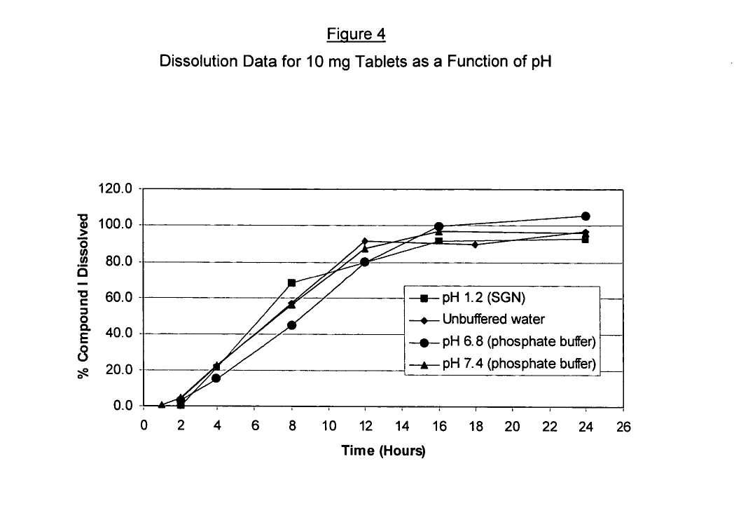

FIG. 4 shows dissolution data for the 10 mg controlled-release formulation of compound (I). The in vitro dissolution of compound (I) from the 10 mg formulation was essentially independent of the pH of the dissolution media.

[image]

[image]

### EXAMPLE 2a

The effect of coating weight on the dissolution of the controlled-release formulations of the invention was determined as follows. The 10 mg dosage formulation tablets comprising compound (I) were prepared as disclosed hereinabove in Example 1a.

The table cores were coated in a Vector LDCS-20 coating pan, and samples were taken at coating levels of 9.4% w/w and 17.2% w/w. The coating components are indicated in Table 8. The ratio of cellulose acetate to PEG-3350 is 9:1 in Example 2a versus 8:2 in Example 1a.

TABLE 8
Coating Formulation

| # | Ingredient | Batch (%) |
|---|---|---|
| 1 | Purified Water | 5.000% |
| 2 | PEG 3350 | 1.000% |
| 3 | Acetone | 85.000% |
| 4 | Cellulose Acetate (398-10) | 9.000% |
| | | 100.000% |

In Vitro Compound (I) Formulations of Example 2a

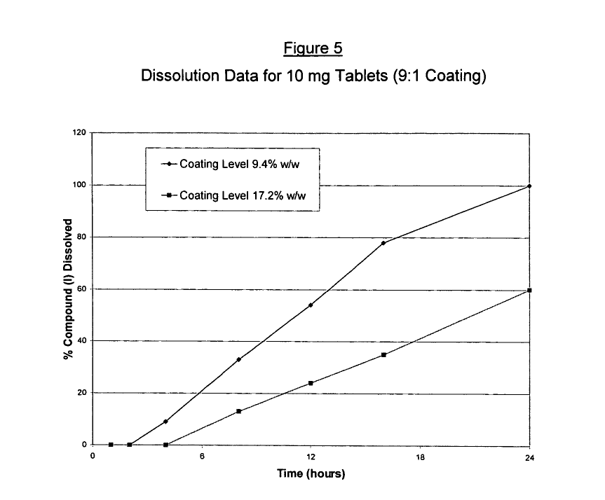

Dissolution of the formulations prepared in Example 2a was determined according to the methodologies of Example 1a. The dissolution profiles are indicated In FIG. 5, and demonstrate that the lag time was greater and the rate of release was slower for the 17.2% coated formulation compared to the 9.4% coated formulation. Furthermore, compared to the 10 mg formulations described in Example 1a, the 9:1 CA/PEG-3350 coating composition resulted in coatings that were less permeable compared to the coatings produced by an 8:2 CA/PEG-3350 coating composition. Similarly, coatings produced by a 7:3 CA/PEG-3350 coating composition were more permeable than an 8:2 CA/PEG-3350 coating composition. These observations about the release rate profiles and permeability of the coatings as a function of the CA/PEG-3350 ratio are consistent with less porous and denser coatings seen with increasing ratio of CA/PEG-3350 by scanning electron microscopy (SEM) analysis.

[image]

### EXAMPLE 3a

Tablets of the SCT controlled-release formulations of the present invention comprising 5 mg of 2-(4-fluorophenoxy)-N-[4-(1-hydroxy-1-methyl-ethyl)-benzyl]-nicotinamide (Ia) were prepared as follows.

Drug-Containing Composition

The drug-containing composition was prepared by combining 2-(4-fluorophenoxy)-N-[4-(1-hydroxy-1-methyl-ethyl)-benzyl]-nicotinamide (Ia), sodium starch glycolate (Explotab; Pennwest Pharmaceuticals; Patterson, N.J.), polyethylene oxide (Polyox WSR-205; Dow Chemical Co.; Midland, Mich.), a granulated mixture of xylitol and sodium carboxymethylcellulose (Xylitab 200; Danisco Sweeteners, Thomson, Ill.), and magnesium stearate. All components with the exception of the magnesium stearate were blended for 20 minutes, sieved through a #30 mesh screen, and blended for an additional 20 minutes. The magnesium stearate was then added followed by blending for an additional four minutes. The formulation components for the drug-containing composition of the 5 mg dosage formulation of compound (Ia) are shown in Table 9 hereinbelow.

TABLE 9
5 mg Tablet
Drug-Containing Composition (layer weight 200 mg)

| | | | Batch | |
|---|---|---|---|---|
| # | Ingredient | Purpose | (%) | mg/tab |
| 1 | Compound (Ia) | PDE4D Inhibitor | 2.50% | 5.00 |
| 2 | Explotab | Swelling Agent | 7.50% | 15.00 |
| 3 | Polyox WSR-205 | Entraining Agent | 44.25% | 88.50 |
| 4 | Xylitab 200 | Diluent/Osmagent | 44.25% | 88.50 |
| 5 | Magnesium | Lubricant | 1.50% | 3.00 |
| | Stearate | | | |
| | | Total | 100.000% | 200.000 |

Water-Swellable Composition

The water-swellable composition was prepared by combining Explotab, microcrystalline cellulose (Avicel PH102; FMC Corp.; Philadelphia, Pa.), lactose Fast-Flo, Red Lake #40 pigment, and magnesium stearate using a blend-screen-blend-lubricate-blend procedure similar to that described hereinabove for the preparation of the drug-containing composition of compound (Ia). The formulation components for the water-swellable composition of the 5 mg dosage formulation of compound (Ia) are shown in Table 10 herein below.

TABLE 10
5 mg Tablet
Water-Swellable Composition (layer weight 100 mg)

| # | Ingredient | Purpose | Batch (%) | mg/tab |
|---|---|---|---|---|
| 1 | Explotab | Swelling | 42.00% | 42.00 |
| | | Agent | | |
| 2 | Avicel PH102 | Tablet Aid | 42.60% | 42.60 |
| 3 | Lactose Fast-Flo | Osmagent | 14.23% | 14.23 |
| 4 | Red Lake #40 | Pigment | 0.15% | 0.15 |
| 5 | Magnesium Stearate | Lubricant | 1.02% | 1.02 |
| | | Total | 100.000% | 100.000 |

Core

Bilayer tablets were prepared by compressing 200 mg of the drug-containing composition comprising compound (Ia) and 100 mg of the water-swellable composition using 11/32″ SRC plain faced tooling on a single-station press to achieve a hardness of about 12 Kp. The 5 mg tablet core weighed about 300 mg.

Coating

The bilayer tablet cores comprising compound (Ia) were coated with a coating solution comprising cellulose acetate (CA 398-10; Eastman Chemical Co.; Kingsport, Tenn.), polyethylene glycol (PEG-3350; Union Carbide Chemical Co.), water, and acetone using an LDCS-20 coating pan. The coating process parameters employed are set forth hereinbelow in Table 11, and the process conditions are indicated hereinbelow in Table 12. The cores were coated to a 10.7% and 21.8% weight gain. Once coated, the tablets were placed in a tray drier overnight at 35° C.

TABLE 11
Coating Process in LDCS-20 Coating Pan

| Process Parameter | Setting |
|---|---|
| Pan Speed | 20 rpm |
| Inlet Air Temperature | 40° C. |
| Outlet Air Temperature | 28° C. |
| Atomization/Pattern Air | 22 psi |
| Spray Rate | 20 g/min |

TABLE 12
Coating Process Conditions for the LDCS-20 Coating Pan

| Process Parameter | Setting |
|---|---|
| Equipment | LDCS-20 coating pan |
| Batch Size | 600-1000 g |
| Pan Speed | 20 rpm |
| Process Air Flow | 30-40 cfm |
| Inlet Air Temperature | 40° C. |
| Outlet Air Temperature | 28° C. |
| Atomization/Pattern Air | 15 psi |
| Spray Rate | 20 g/min |

Delivery Ports

A total of five holes, each 0.9 mm in diameter, were mechanically drilled on the drug-containing composition side of the tablet using a drill press with a 0.9 mm drill bit.

In Vitro Performance of Compound (Ia) Formulations

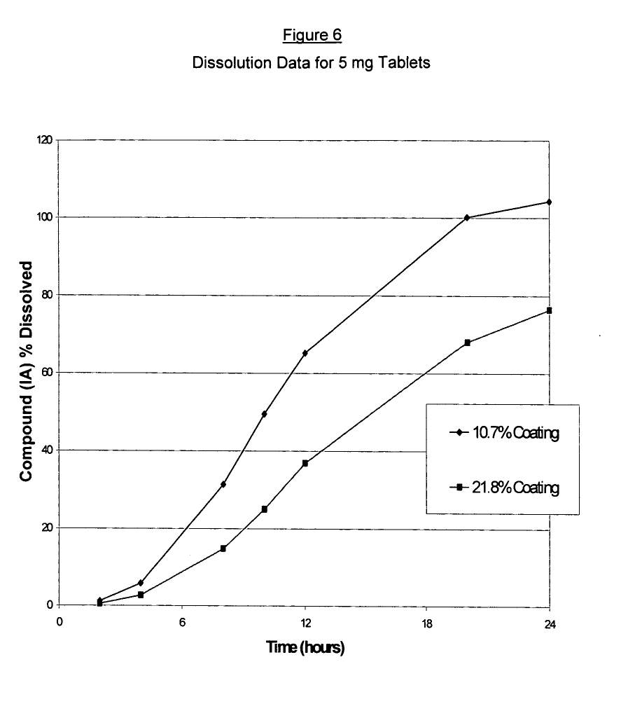

Dissolution of the 5 mg SCT controlled-release formulations of compound (Ia) was determined using a USP Apparatus II (rotating paddles at 100 rpm in 900 mL of potassium phosphate buffer at pH 7.5). The amount of compound (Ia) dissolved was determined by HPLC using UV-VIS detection. The dissolution profile exhibited about a three-hour lag time followed by release of compound (Ia) at a rate that was directly dependent on the coating level. FIG. 6 depicts the average release profile for the 5 mg formulations.

[image]

[image]

In Vivo Nausea/Emesis Plasma Profiles

In order to demonstrate the utility of the instant SCT controlled-release formulations in the reduction of nausea and/or emesis induced by PDE4D inhibitor treatment, human in vivo oral dosage escalation studies were performed employing OPC (oral powder for constitution) immediate-release (1R) formulations and the SCT controlled-release formulations of the instant invention comprising compound (I). The results of these studies are summarized in Tables 13 and 14 hereinbelow. In Tables 13 and 14, standard deviation values are reported parenthetically. Furthermore, a “Yes” entry means greater than half of the subjects experienced the indicated side-effect, while a “No” entry indicates none, or less than half, of the subjects experienced the indicated side-effect.

The 3 mg dosages of compound (I) indicated in Tables 13 and 14 comprised the simultaneous administration of three 1 mg tablets of the SCT formulations of the present invention. Likewise, the 30 mg dosages of compound (I) comprised the simultaneous administration of three 10 mg tablets of the SCT formulations of the present invention.

The OPC immediate-release formulations comprising compound (I) were prepared extemporaneously by diluting powdered sodium citrate with water to a final concentration of 0.02 M and dissolving compound (I) therein with stirring. The prepared solution was then orally administered to the subject.

The mean peak plasma concentrations (Cₘₐₓ) and the time such peak concentrations were attained (Tₘₐₓ) following administration of the immediate-release formulations of compound (I) are set forth hereinbelow in Table 13. Analysis of such data will establish the relationship between the plasma pharmacokinetics and nausea and emesis side-effects in human subjects administered with dosages of compound (I). At Cₘₐₓ values of less than 60 ng/mL, compound (I) was generally well tolerated. At dosages of 3 mg and above given in the fasted state, or 10 mg and above in the fed state, there was an onset of nausea indicating that the threshold for nausea occurs at about 60 ng/mL. Similarly, at dosages of 10 mg in the fasted state, or 30 mg in the fed state, corresponding to Cₘₐₓ values of about 200 ng/mL, there was significant occurrence of emesis indicating that the threshold for emesis is about 200 ng/mL. Dosages of compound (I) greater than 10 mg in the fasted state, and 30 mg in the fed state, could not be successfully administered due to the severity of the resulting nausea and emesis.

TABLE 13
Plasma Concentration (Cₘₐₓ) vs. Time (Tₘₐₓ) Profiles for
Immediate-Release Formulations of Compound (I)

| Dose and | | | | | |
|---|---|---|---|---|---|
| Fed/fasted | Cₘₐₓ | Tₘₐₓ | AUC | | |
| Condition | (ng/ml) | (hr) | (ng · hr/ml) | Nausea | Emesis |
| 0.1 mg | 2.72 | 1.4 | 7.03 | No | No |
| fasted | (0.961) | (0.4) | (3.47) | | |
| 0.3 mg | 8.72 | 1.42 | 42.2 | No | No |
| fasted | (2.51) | (0.5) | (16.1) | | |
| 1 mg | 27.6 | 1.3 | 151 | No | No |
| fasted | (8.56) | (0.3) | (64) | | |
| 3 mg | 62.9 | 1.1 | 358 | Yes | No |
| fasted | (14.3) | (0.2) | (61.1) | | |
| 3 mg fed | 25.0 | 3.8 | 300 | No | No |
| | (7.17) | (0.4) | (62) | | |
| 10 mg | 206 | 1.2 | 1170 | Yes | Yes |
| fasted | (86.6) | (0.3) | (621) | | |
| 10 mg fed | 61.1 | 3.2 | 851 | Yes | No |
| | (8.09) | (0.8) | (186) | | |
| 30 mg fed | 201 | 1.4 | 2040 | Yes | Yes |
| | (51.5) | (0.8) | (1390) | | |

The mean peak plasma concentrations (Cₘₐₓ) and the time such peak concentrations were attained (Tₘₐₓ) following administration of the instant SCT controlled-release formulations of compound (I) are set forth hereinbelow in Table 14. The data demonstrates that the instant SCT formulations provide plasma concentrations significantly higher than the previously established thresholds for both nausea and emesis for the IR formulations. In some subjects given the 30 mg dosage in the fed state, the peak concentrations were in excess of 250 ng/mL, with the mean being 198 ng/mL. Predicated upon a Cₘₐₓ threshold of 200 ng/mL for emesis, a much higher incidence of emesis would have been predicted to occur at this dosage. At these concentrations however, there was some incidence of nausea, however, no emesis was observed.

TABLE 14
Plasma Concentration (Cₘₐₓ) vs. Time (Tₘₐₓ) Profiles for
SCT Formulations of Compound (I)

| Dose and | | | | | |
|---|---|---|---|---|---|
| Fed/fasted | Cₘₐₓ | Tₘₐₓ | AUC | | |
| Condition | (ng/ml) | (hr) | (ng · hr/ml) | Nausea | Emesis |
| 1 mg fasted | 2.63 | 5.36 | 23.4 | No | No |
| | (1.39) | (2.77) | (18.6) | | |
| 3 mg fasted | 7.37 | 4.92 | 110 | No | No |
| | (4.04) | (1.88) | (70.6) | | |
| 10 mg | 24.9 | 5.17 | 329 | No | No |
| fasted | (13.0) | (2.72) | (170) | | |
| 10 mg fed | 59 | 11.2 | 954 | No | No |
| | (18) | (1.8) | (319) | | |
| 30 mg | 61 | 10.4 | 1330 | No | No |
| fasted | (42.4) | (4.42) | (771) | | |
| 30 mg CR | 142 | 10.8 | 2240 | No | No |
| low fat | (68) | (3.5) | (1210) | | |
| 30 mg high | 198 | 11.5 | 3230 | Yes | No |
| fat | (70.3) | (2.84) | (989) | | |
| 20 mg high | 137 | 12 | 2480 | No | No |
| fat | (51.3) | (3.46) | (1020) | | |

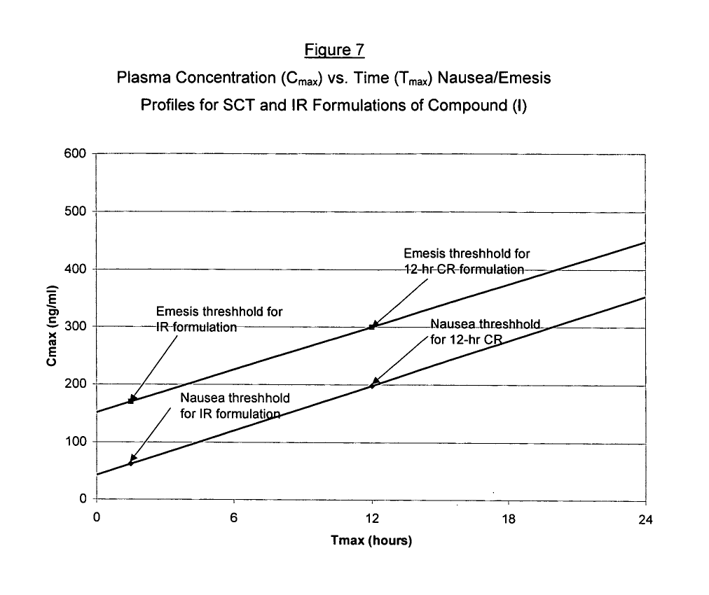

The Tₘₐₓ values observed with the instant SCT formulations comprising compound (I) are also much longer, with mean values ranging from about 10.4 to about 12 hours. These observations are consistent with a prolonged release and prolonged absorption of compound (I). Thus, it is found that the thresholds for nausea and emesis can be increased when the Tₘₐₓ is increased from about 1.5 hours to greater than about 10 hours. The exact relationship between the Cₘₐₓ, Tₘₐₓ, and the onset of nausea and/or emesis is not precisely known, however, as shown in FIG. 7, a linear increase can be reasonably assumed.

[image]

It can be further concluded that the rate of rise of plasma concentrations of compound (I) is a critical factor that determines PDE4D toleration, and the Cₘₐₓ to Tₘₐₓ ratio, defined in ng/ml/hr., can approximate this rate of rise of plasma concentrations. From the IR formulation data hereinabove, it may be concluded that a Cₘₐₓ/Tₘₐₓ ratio of less than about 40 will maintain a low incidence of nausea, and a Cₘₐₓ/Tₘₐₓ ratio of less than about 100 will maintain a low incidence for emesis. Since the threshold for nausea is less than that for emesis, a reduction in nausea concomitantly reduces emesis. The lowest Cₘₐₓ/Tₘₐₓ ratio embraced within the scope of the invention is about 15, with a preferred ratio being between about 15 to about 40. A Cₘₐₓ/Tₘₐₓ ratio of between about 15 and about 30 is especially preferred. Ratios higher than about 15 to about 40 are also presently believed to be useful in that, while they may not reduce the incidence of nausea, they may reduce the incidence of emesis, which is currently considered advantageous.

Therefore, a controlled-release formulation that results in a Tₘₐₓ greater than about 1.5 hours will afford higher plasma concentration thresholds for both nausea and emesis compared to those observed for an immediate release formulation. In fact, for every hour increase in Tₘₐₓ above 1.5 hours, the thresholds for nausea and emesis increase by 13 ng/mL. Thus, for a Tₘₐₓ of six hours, the nausea and emesis thresholds are estimated to be about 120 ng/mL and about 228 ng/mL, respectively. For a Tₘₐₓ of 12 hours, the nausea and emesis thresholds are estimated to be about 198 ng/mL and about 306 ng/mL, respectively. For a Tₘₐₓ of 18 hours, the nausea and emesis thresholds are estimated to be about 276 ng/mL and about 384 ng/mL, respectively.

It has been further demonstrated that a controlled-release formulation having an in vitro release duration of about 12 hours results in a Tₘₐₓ of 12 hours, which is consistent with good in vitro—in vivo correlations observed for SCT controlled-release formulations. Thus, in order to improve the side-effect profiles of nausea and emesis, the in vitro delivery duration of the controlled-release formulation should be greater than about 1.5 hours. Although controlled-release formulations with in vivo delivery durations of 24 hours, or higher, can be designed, delivery durations longer than about 18 hours are generally not preferred because of the limited gastrointestinal transit time (mouth to anus transit) of the dosage formulation. An in vivo delivery duration of greater than about six hours is generally preferred. An in vivo delivery duration of between about 12 hours and about 18 hours is especially preferred. The phrase “delivery duration” denotes the time at which greater than about 80% of the initial drug load is released in vivo from the dosage form.

Without being limited to a specific theory, hypothesis, or mechanism, it is presently believed that the improvements in the nausea and emesis side-effect profiles observed with the instant SCT formulations comprising PDE4D inhibitor (I) are at least partially related to the in vivo time lag before onset of PDE4D delivery, which is believed to result in the lower concentrations of the PDE4D inhibitor in the upper GI tract, most notably in the stomach and/or duodenum. The present SCT controlled-release formulations exhibit both an in vitro and an in vivo time lag.

The phrase “in vivo time lag” refers generally to the difference in the average delay between drug administration and the beginning of the absorption phase for the dosage form of the present invention and an immediate-release dosage form, such as a solution, a suspension, or conventional fast-dissolving tablets or capsules. The in vivo time lag is calculated from the plasma concentration time profiles obtained after a single dose study in at least six subjects using deconvolution techniques, method of residuals, or mass balance methods, all of which will be well-known to one of ordinary skill in the art. See, for example, Wagner, et al., J. Pharm. Sci., 53, 1392-1394 (1964), or M. Rowland and T. N. Tozar, “Clinical Pharmacokinetics: Concepts and Applications, 3rd Edition”, Lippincott, Williams and Wilkins (1995). The “in vivo (plasma) time lag” refers to the delay between administration of the formulation to an animal or human, and the appearance of drug in the plasma. This time lag is estimated from the plasma concentration vs. time profile by extrapolating the first few time points when the plasma concentrations are increasing back to the x-axis (time axis). Generally preferred in vivo time lags are in the range of from about 0.5 to about 4, hours, preferably from about two to about four hours.

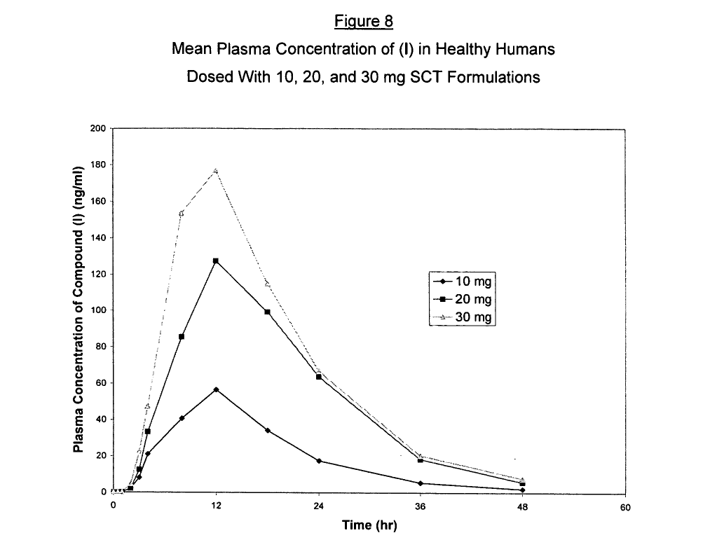

Mean plasma concentrations of compound (I) in healthy human volunteers dosed in the fed state with 10, 20, and 30 mg SCT formulations comprising compound (I) are shown hereinbelow in FIG. 8.

[image]

[image]

The in vivo time lag ranges from between about 0.5 hours to about four hours, preferably from between about 0.5 hours to about two hours. In vivo time lag values significantly greater than about four hours may result in a lower total exposure with the controlled-release formulation, compared to the same dosage administered as an IR formulation.

The phrase “in vitro time lag” is the delay between the time of introduction of the SCT formulation into the dissolution media, and the initiation of release of the PDE4D inhibitor, or the pharmaceutically acceptable salt thereof, therefrom. The release is considered to be initiated when about 10% by weight, but preferably about 5% by weight, of the drug is released from the dosage form. In vitro time lags may be calculated from the in vitro dissolution test data using known methods. In vitro time lags of the instant SCT formulations comprising compounds (I) and (Ia) are depicted graphically in FIGS. 1 through 6 hereinabove.

1. A controlled-release pharmaceutical formulation comprising a phosphodiesterase type 4D (PDE4D) inhibitor, or a pharmaceutically acceptable salt thereof, said formulation exhibiting at least one of the following characteristics:
(i) a Tₘₐₓ of greater than about 1.5 hours when dosed in vivo;
(ii) less than about 80% of said PDE4D inhibitor, or said pharmaceutically acceptable salt thereof, is released in vivo at about 1.5 hours;
(iii) less than about 80% of said PDE4D inhibitor, or said pharmaceutically acceptable salt thereof, is released in vitro at about 1.5 hours;
(iv) an in vitro delivery lag time prior to initiation of release of said PDE4D inhibitor, or said pharmaceutically acceptable salt thereof, of between 0.5 hours and about four hours;
(v) an in vivo delivery lag time prior to initiation of release of said PDE4D inhibitor, or said pharmaceutically acceptable salt thereof, of between 0.5 hours and about four hours.

2. A controlled-release formulation of claim 1, wherein said PDE4D inhibitor comprises (R)-2-[4-({[2-(benzo[1,3]dioxol-5-yloxy)-pyridine-3-carbonyl]-amino}-methyl)-3-fluoro-phenoxy]-propionic acid, or a pharmaceutically acceptable salt thereof, or 2-(4-fluorophenoxy)-N-[4-(1-hydroxy-1-methyl-ethyl)-benzyl]-nicotinamide, or a pharmaceutically acceptable salt thereof.

3. A controlled-release pharmaceutical formulation of claim 1 comprising a core, and a coating around said core, wherein:
(a) said core comprises a drug-containing composition and a water-swellable composition, each occupying essentially separate regions within said core;
(b) said drug-containing composition comprises a PDE4D inhibitor, or a pharmaceutically acceptable salt thereof, and a drug-entraining agent;
(c) said water-swellable composition comprises a swelling agent; and
(d) said coating is water-permeable, water-insoluble, and has at least one delivery port therethrough.

4. A formulation of claim 3, wherein said PDE4D inhibitor comprises (R)-2-[4-({[2-(benzo[1,3]dioxol-5-yloxy)-pyridine-3-carbonyl]-amino}-methyl)-3-fluoro-phenoxy]-propionic acid, or a pharmaceutically acceptable salt thereof, or 2-(4-fluorophenoxy)-N-[4-(1-hydroxy-1-methyl-ethyl)-benzyl]-nicotinamide, or a pharmaceutically acceptable salt thereof.

5. A formulation of claim 3, wherein said swelling agent of said water-swellable composition comprises an ionic or non-ionic polymeric swelling agent.

6. A formulation of claim 5, wherein said ionic polymeric swelling agent is selected from the group consisting of polacrilin potassium, polyacrilin resins, sodium alginate, sodium croscarmellose, and sodium starch glycolate; and said non-ionic polymeric swelling agent is selected from the group consisting of alginic acid, carboxymethylcellulose, hydroxypropylcellulose, microcrystalline cellulose, crospovidone, methylcellulose, polyethylene oxide, povidone, and starch.

7. A formulation of claim 6, wherein said swelling agent is selected from the group consisting of polyethylene oxide, sodium starch glycolate, and sodium croscarmellose.

8. A formulation of claim 3, wherein said water-swellable composition further comprises an osmotically effective agent.

9. A formulation of claim 3, wherein said water-swellable composition further comprises a tableting aid.

10. A formulation of claim 3, wherein the mass ratio of said drug-containing composition to said water-swellable composition has a value of at least 1.0, and said core has a strength following tableting of at least 3 Kp/cm².

11. A formulation of claim 3, wherein said coating has a water flux (40°/75% relative humidity) of at least 1.0×10⁻³ gm/cm² hr, and a durability of at least 1 Kp/cm².

12. A formulation of claim 3, wherein said drug-containing composition further comprises a fluidizing agent, said fluidizing agent having a solubility of at least 30 mg/mL and comprising at least 10 wt % of said drug-containing composition.

13. A formulation of claim 12, wherein said fluidizing agent is selected from the group consisting of an organic acid, an organic base, and a sugar.

14. A formulation of claim 3, wherein said drug-containing composition further comprises a solubilizer.

15. A formulation of claim 14, wherein said solubilizer is selected from the group consisting of an organic acid, an organic base, an inorganic acid, an inorganic base, a surfactant, and a cyclodextrin.

16. A formulation of claim 3, wherein said drug-containing composition further comprises a concentration-enhancing polymer.

17. A formulation of claim 16, wherein said concentration-enhancing polymer selected from the group consisting of:
(a) ionizable cellulosic polymers;
(b) non-ionizable cellulosic polymers; and
(c) vinyl polymers and copolymers having substituents selected from the group consisting of hydroxyl, alkylacyloxy, and cyclicamido.

18. A formulation of claim 3, wherein said PDE4D inhibitor, or said pharmaceutically acceptable salt thereof, is in the form of a solid, amorphous dispersion.

19. A formulation of claim 18, wherein said solid, amorphous dispersion comprises a solid dispersion of said PDE4D inhibitor, or said pharmaceutically acceptable salt thereof, in a concentration-enhancing polymer.

20. A formulation of claim 3 wherein said PDE4D inhibitor comprises (R)-2-[4-({[2-(benzo[1,3]dioxol-5-yloxy)-pyridine-3-carbonyl]-amino}-methyl)-3-fluoro-phenoxy]-propionic acid, or a pharmaceutically acceptable salt thereof; and wherein said swelling composition comprises between about 50% and about 75% w:w of the swelling layer being polyethylene oxide of a molecular weight (weight average) of between about 1,000,000 and about 7,000,000, and between about 25% and about 50% w:w of sodium chloride; and said drug composition comprises between about 0.5 and about 10% (w:w) of said drug, about 50% and about 70% of polyethylene oxide having a weight average molecular weight of between about 100,000 and about 600,000; and said coating comprises cellulose acetate and polyethylene glycol having a molecular weight (weight average) of between about 1,000 and about 10,000.

21. A formulation of claim 3 wherein said PDE4D inhibitor comprises 2-(4-fluorophenoxy)-N-[4-(1-hydroxy-1-methyl-ethyl)-benzyl]-nicotinamide or a pharmaceutically acceptable salt thereof; and wherein said swelling composition comprises about 35 and about 45% (w:w) sodium starch glycolate, about 35% and about 45% microcrystalline cellulose and about 10% and about 20% lactose; said drug layer comprises about 0.5 and about 10% of said drug, about 5% and about 10% sodium starch glycolate, about 30% and about 50% polyethylene oxide having a weight average molecular weight of about 200,000 and about 1,000,000, and about 30% and about 55% xylitol; and said coating comprises cellulose acetate and polyethylene glycol having a molecular weight (weight average) of between about 1,000 and about 10,000.

22. A method of treating disorders and conditions mediated by the PDE4D isozyme, which comprises administering to a mammal in need of such treatment a therapeutically effective amount of a PDE4D inhibitor, or a pharmaceutically acceptable salt thereof, in a controlled-release formulation of claim 3.

23. A method of claim 22, wherein said PDE4D inhibitor comprises (R)-2-[4-({[2-(benzo[1,3]dioxol-5-yloxy)-pyridine-3-carbonyl]-amino}-methyl)-3-fluoro-phenoxy]-propionic acid, or a pharmaceutically acceptable salt thereof, or 2-(4-fluorophenoxy)-N-[4-(1-hydroxy-1-methyl-ethyl)-benzyl]-nicotinamide, or a pharmaceutically acceptable salt thereof.

24. A method of claim 22, wherein said disorders and conditions are selected from the group consisting of:
(a) inflammatory diseases and conditions selected from the group consisting of joint inflammation, rheumatoid arthritis, rheumatoid spondylitis, osteoarthritis, inflammatory bowel disease, ulcerative colitis, chronic glomerulonephritis, dermatitis, and Crohn's Disease;
(b) respiratory diseases and conditions selected from the group consisting of asthma, acute respiratory distress syndrome, chronic obstructive pulmonary disease, bronchitis, chronic obstructive airway disease, and silicosis;
(c) infectious diseases and conditions selected from the group consisting of sepsis, septic shock, endotoxic shock, gram negative sepsis, toxic shock syndrome, fever and myalgias due to bacterial, viral or fungal infection, and influenza;
(d) immune diseases and conditions selected from the group consisting of autoimmune diabetes, systemic lupus erythematosis, graft v. host reaction, allograft rejections, multiple sclerosis, psoriasis, and allergic rhinitis; and
(e) other diseases and conditions selected from the group consisting of bone resorption diseases, reperfusion injury, cachexia secondary to infection or malignancy, cachexia secondary to human immunodeficiency syndrome (AIDS), human immunodeficiency virus (HIV), infection, or AIDS related complex (ARC), keloid formation, scar tissue formation, Type 1 diabetes mellitus, and leukemia.

25. A method of claim 24, wherein said disorders and conditions are selected from the group consisting of asthma, acute respiratory distress syndrome, chronic obstructive pulmonary disease, bronchitis, chronic obstructive airway disease, and silicosis.

26. A method of reducing PDE4D inhibitor treatment-induced nausea and/or emesis in a mammal which comprises administering said PDE4D inhibitor, or a pharmaceutically acceptable salt thereof, to said mammal in the form of a controlled-release formulation of claim 3.

27. A method of claim 26, wherein said PDE4D inhibitor comprises (R)-2-[4-({[2-(benzo[1,3]dioxol-5-yloxy)-pyridine-3-carbonyl]-amino}-methyl)-3-fluoro-phenoxy]-propionic acid, or a pharmaceutically acceptable salt thereof, or 2-(4-fluorophenoxy)-N-[4-(1-hydroxy-1-methyl-ethyl)-benzyl]-nicotinamide, or a pharmaceutically acceptable salt thereof.
